(19) United States
(12) Patent Application Publication
(10) Pub. No.: US 2015/0025311 A1
(43) Pub. Date: Jan. 22, 2015

(54) Small single-port arthroscopic lavage, directed tissue drying, biocompatible tissue scaffold and autologous regenerated cell placement delivery system

(71) Applicants: Jeffrey S. Kadan, Laguna Niguel, CA (US); Ronald Coleman, Oakland, CA (US)

(72) Inventors: Jeffrey S. Kadan, Laguna Niguel, CA (US); Ronald Coleman, Oakland, CA (US)

(21) Appl. No.: 13/999,743

(22) Filed: Mar. 19, 2014

Related U.S. Application Data
(60) Provisional application No. 61/958,879, filed on Mar. 15, 2013.

(51) Int. Cl. A61B 1/317; A61B 17/34; A61F 2/46; A61B 1/018; A61B 1/07; A61B 1/06; A61B 1/00; A61M 1/00

(52) U.S. Cl. CPC A61B 1/317; A61B 1/00068; A61B 17/3421; A61B 17/3468; A61M 1/0035; A61M 1/0064; A61B 1/00105; A61B 1/00135; A61B 1/018; A61B 1/07; A61B 1/0684; A61F 2/461; A61B 2017/00238
USPC 600/104

(57) ABSTRACT

A system for performing arthroscopic lavage, directed tissue drying, and the accurate placement of a biocompatible tissue scaffold for the adherence of autologous regenerated cells through a small single port of entry into a joint compartment. The system is comprised of a handpiece having valves for irrigation and suctioning and a dual valve swivel cannula attached to the handpiece. The system includes a mobile cart, high resolution camera, light source, optical coupler, high-resolution monitor, an air compressor to power individually controlled irrigation pumps to deliver irrigation fluid to a handpiece and a vacuum suction console to collect fluid. The system also includes an insufflator to maintain distension immediately following the lavage and to dry tissue in preparation for directed tissue scaffold and regenerative cell placement. The delivery system achieves accurate biocompatible tissue scaffold placement to a specific tissue site or sites within the joint utilizing a small diameter arthroscope for direct visualization while inserting and advancing a grasping instrument or device through one of two valves located on the cannula. While holding the tissue scaffold in the jaws of the grasping device, it is advanced through the cannula lumen and extended beyond the distal tip and placed on the dried tissue site. Removing the grasping device, a catheter is then inserted and advanced through a cannula valve into the lumen and extended beyond the distal tip to the scaffold placed and prepared tissue site. A means of applying torque to the catheter tip further enhances the ability for accurate, exact placement of cells to a specific scaffold receptive tissue site. The cells are then injected into and through the catheter and applied under direct visualization to the scaffold. As comprised, the small single-port system allows a physician to perform the diagnosis, clean the joint space of debris and degradative enzymes using pressurized irrigation and suction, followed by a rapid conversion from a sterile saline fluid distension media to a dry gas CO2 distension media and directed tissue drying, and the accurate placement of a biocompatible tissue scaffold for the adherence and accurate placement of regenerated cells through a catheter to a specific tissue site within a joint.

### BACKGROUND OF THE INVENTION

1. Field of the Invention

This invention relates to systems to perform arthroscopies of joints such as the knee and more particularly relates to an arthroscopic lavage system for performing arthroscopies to clean debris and degradative enzymes from a joint. In addition this invention also relates to the field of autologous regenerative cell healing therapies utilizing an arthroscopic approach to better facilitate cell placement. The system combines distension and thorough cleaning of the joint by means of pressurized ‘wet’ saline fluid inflow and vacuum suction outflow, and continued distension and directed tissue drying by means of controlled and regulated ‘dry’ CO2 gas inflow while maintaining vacuum suction outflow. This regenerative cell delivery system also includes a small grasping device for precise manipulation and placement of a biocompatible tissue scaffold to a dried tissue site, and a small catheter with a controllable distal tip for the precise and accurate placement of autologous regenerated cells to either a dried tissue site or to a previously affixed tissue scaffold.

A steerable catheter specifically designed for cell delivery and for use with single-port needle arthroscopy procedures. The catheter is advanced through a port in the hand-piece to deliver cells and cellular products/agents to joints or other anatomic targets. The body of the catheter is long and tubular and contains an inner lumen; has a distal end as well as a proximal end that incorporates a handle for control by the operator. The device uses catheter technology and methods for precise and targeted delivery of cell products to joints, muscles, nerves, bone, tendons, and ligaments in order to repair or perhaps reverse the effects of injury. Cell products may include platelet rich plasma (PRP), growth factors or super concentrated platelets (SCP). A catheter has been developed that reduces the number of insertion trajectories required for cell delivery. The invention could therefore significantly lower operating time and reduce surgical risk. Catheter-based injections are less invasive and make it possible to administer cell products used as sole or multiple interventions. All interventions are accomplished while under direct visualization and through only 1 small port of entry into the joint.

2. Background Information

Arthroscopy is a surgical procedure in which an endoscope (arthroscope) is inserted into a joint. Fluid is then injected into the joint to slightly distend the joint and allow visualization of structures within the joint. Surgery is usually viewed on a monitor so that the whole operating team can visualize the surgical procedure that is being performed. The arthroscopy procedure falls into two types; operative and diagnostic.

Operative arthroscopy is more interventional, utilizing larger devices and multiple ports to accomplish a variety of procedures designed to repair internal derangement or tears of intra-articular structures. Diagnostic arthroscopy is less invasive, requiring smaller devices and a single port of entry into the joint. Operative arthroscopes are typically four (4) mm in diameter. The operative arthroscopic procedure is often conducted under general anesthesia and is used to examine and treat the inside of the joint for damaged tissue. Most common types of surgery using operative arthroscopic procedures includes the removal or repair of torn meniscus (cartilage), ligament and tendon reconstruction, removal of loose debris and trimming or shaving damaged articular cartilage. Diagnostic arthroscopy is done under local anesthetic only and is most often accompanied by a thorough rinsing out of the joint (lavage).

The value of arthroscopy as a diagnostic and therapeutic tool is well recognized by physicians. Recent advances have made it technically feasible to perform diagnostic or single-port needle arthroscopy procedures in a physician's office using a small, 1.7 mm fiberoptic arthroscope. Generally the single-port arthroscopic lavage procedure is used to diagnose and evaluate joint pathology, as well as relieve pain and limited range of motion symptoms from osteoarthritis that is not relieved by traditional, conservative medical treatment and management. It is also utilized in treating refractory synovitis and determining uncertain etiology.

The small single-port procedure has also been found to be an excellent alternative for those patients unwilling and/or unable to tolerate the increased risks of general anesthesia and major, invasive joint replacement surgery. Osteoarthritis (OA) is a common problem for many middle-aged and elderly people. OA is sometimes referred to as degenerative or wear-and-tear arthritis, a byproduct of the aging process. It can also result from direct injury or trauma to a joint. Instability from ligament damage and/or meniscal injuries causes abnormal wear and tear of the cartilage within a joint. Not all cases of osteoarthritis are related to prior injury however. Research has shown that many are prone to develop osteoarthritis and the tendency is genetic. Obesity is also a contributory factor.

The main problem of osteoarthritis is degeneration of the cartilage that covers articulating surfaces of the joint, resulting in areas where bone rubs against bone creating bone spurs. Generally osteoarthritis develops slowly over several years. The symptoms are mainly pain, swelling, and stiffening of the joint. As the condition worsens or progresses, pain can interfere with simple, daily activities. Traditional conservative methods of medical treatment include taking anti-inflammatory medication, heat, ice, physical therapy and cortisone injections to reduce the swelling and inflammation of the joint and a variety of pain medications suppress the body's systemic pain response. Intra-articular injections of hyaluronic acid, a natural high-density viscous fluid and lubricant acts as a synthetic synovial fluid and is also routinely used by physician's in fighting the debilitating effects of OA.

The emergence of autologous regenerative cell healing therapies such as PRP, platelet enriched plasma, and stem cells have demonstrated a treatment paradigm in which patients seek less-invasive, more natural therapies in place of far more invasive operative surgeries and joint replacements. The majority of regenerated cell procedures are routinely done utilizing high definition ultrasound or C-arm guidance into the joint for the injection of the regenerated cells.

Single-port arthroscopic surgeries have been performed in the doctor's office and ambulatory surgery centers to diagnose and treat a variety of symptoms including osteoarthritis, rheumatoid arthritis, crystal-induced arthritis, and pain of unknown etiology. Routinely performed under local anesthetic, the patient remains awake throughout the procedure. A video monitor is typically used by the physician while performing the procedure, and if desired, the patient may observe as well.

This invention is a system that combines the benefits of directly visualizing the tissue being treated while using arthroscopic lavage to thoroughly clean the entire joint, as well as dry and prepare a specific tissue site for the accurate, precise placement and adhesion of autologous regenerated cells for superior, long lasting patient outcomes. The procedure is performed under sterile conditions to minimize the possibility of infection, and the patient is prepared and draped in the usual fashion.

The joint entry site or portal is identified and local anesthesia is injected into the entry site and surrounding tissue. Local anesthesia is also injected inside the joint capsule. An alternative to local anesthetic is the application of an epidural block.

A small 5 mm entry site incision is made with a #11 scalpel and the cannula with attached handpiece and sharp trocar locked in place is inserted to the level of the joint capsule. The sharp trocar is then removed and replaced with a blunt obturator to avoid damaging interior joint space tissue when the cannula is ‘popped’ through the joint capsule and into the joint space. When cannulation is achieved the blunt obturator is then removed from the cannula and the 1.7 mm arthroscope is inserted and locked in place. Irrigation or inflow is performed through the cannula, attached handpiece and the irrigation valve on the handpiece which is connected by tubing to a hanging bag of irrigation solution (sterile saline) under pressure. Infusion of saline is performed until distension and a clear visual field is obtained and is intermittently maintained throughout the procedure by alternate use of both the irrigation and suction valves.

With the arthroscope inserted in the cannula and a clear visual field, the compartments of the joint may be visualized, inspected and accurately diagnosed. The irrigation or lavage of the joint and subsequent aspiration or removal of fluid removes particulate matter and loose bodies floating in the joint and has been clinically documented in having beneficial effects with regard to pain relief.

The small single-port flushing of diseased synovial fluid depleted of its' natural high viscosity shock absorbing properties, along with the pain producing degradative enzymes and irritants, byproducts and facilitators of the OA disease progression, has been documented to inhibit and retard the ongoing disease cycle providing significant symptomatic pain relief.

By comparison, operative arthroscopy requires a minimum of two, three, or more larger ports into the joint for greater access required for the instrumentation necessary to perform far more invasive procedures. A disadvantage of the present system of operative arthroscopy is the requirement for additional portals for insertion of surgical instruments and the increased level of insult to the body due to the higher degree of invasiveness.

It is therefore one object of the present invention to provide an arthroscopic lavage system using a small single port entry allowing the physician to use a minimally invasive, direct visualization approach under local anesthetic for diagnosis and also provide therapeutic benefit of complete flushing and cleansing of the joint with sterile saline (lavage).

Another object of the present invention is to provide a unique proprietary suction/irrigation handpiece that doubles as an entry cannula into the joint, and also as the housing for a 1.7 mm fiberoptic arthroscope during the procedure. The handpiece also provides vacuum suction and pressurized irrigation capabilities on demand through finger controlled trumpet valves.

Another object of the present invention is to provide a small single-port arthroscopic lavage system that permits diagnostic evaluation of a joint along with therapeutic lavage which provides long-term pain reduction/relief by flushing loose bodies and the chemical irritants commonly found in chronic osteoarthritis (OA) and rheumatoid arthritis (RA).

Yet another object of the present invention is to provide an arthroscopic lavage system that uses devices of very small size and a single entry port that is an advantage over multiple punctures and larger ports used in standard operative arthroscopy, making the procedure ideal for use in an office or clinic setting. With the system disclosed and described, arthroscopic lavage may be performed under local anesthetic and in conjunction with a mild oral sedative. Patients experience minimum discomfort and generally return to normal activities within 24-48 hours.

Still another object of the present invention is to provide an arthroscopic lavage system as an alternative to magnetic resonance imaging (MRI) for diagnosing joint disease and derangement.

The arthroscopic lavage procedure is both diagnostic and therapeutic while the MRI is only diagnostic and does not permit the opportunity to visualize joint pathology directly or allow for any interventional treatment modalities.

Direct visualization of joint surfaces and pathology is clinically documented as being a superior form of diagnostic modality when compared to MRI and is also another object of the invention.

Still another object of the present invention is to provide an arthroscopic lavage procedure that allows a number of patients, particularly the elderly and those with heart disease, compromised respiratory function and diabetes that are not candidates for traditional operative procedures due to the added risk of general anesthesia, to be treated. The system of the present invention provides those patients who have failed conservative medical management and are unwilling or unable to undergo total or partial joint replacement, a minimally invasive alternative with a high rate of clinically documented success.

Additionally, another object of the present invention of an arthroscopic lavage and regenerative cell delivery system is the addition, and substitution of ‘dry’ CO2 gas for use immediately following the ‘wet’ saline lavage. Gas flow utilizes the existing irrigation/inflow circuit by replacing the air compressor and irrigation pump with an insufflator attached directly to the irrigation/inflow tubing, while the suction/outflow circuit remains unchanged. The dual purpose of maintaining distension and the ability to also perform directed tissue drying are made possible by incorporating use of an insufflator in the inflow circuit.

Further, another object of the present invention in the field of regenerative cell healing therapies is an arthroscopic lavage and regenerative cell delivery system incorporating the addition of a small grasping instrument or device designed specifically for the purpose of precisely placing and affixing a biocompatible tissue scaffold to a dried tissue site. The grasping instrument/device is inserted and advanced through an open auxiliary valve into and through the cannula and advanced to the tissue site under constant, direct visualization. And still another object of the present invention in the field of regenerative cell healing therapies is an arthroscopic lavage and regenerative cell delivery system incorporating the addition of a special catheter designed specifically for the purpose of precisely placing autologous cells directly on a dried tissue site, or directly to a biocompatible scaffold previously affixed to a dried tissue site. The catheter tip will also incorporate the ability to control the angle of approach to the tissue site enabling a more precise, accurate placement of regenerated cells to the target area while under direct visualization.

### Cell Delivery to ACL

Studies of cell delivery efficiency have shown that even in easier to inject joints like the knee, experienced physicians injecting blindly can miss the joint from 14-45% of the time (depending on approach and the severity of condition). This means that injecting without imaging guidance (blind) will deliver stem cells into the proper space in knee joint only about half the time in some patients. Stem cells that aren't in the target area of the knee will have no effect on the knee. The accuracy in other joints is often worse because the joints are smaller or have more soft-tissue. Recent studies compared effectiveness of stem cells placed blindly into the joint (intra-articular) versus exactly on the lesion (local adherent). A control was just an injection of saline. Control samples showed little to no change after injection as did intra-articular blind injections. When cells were placed directly on a specific damaged spot within the joint, significant cartilage repair was generally observed by comparison. The same holds true for injecting cells into a specific tendon, ligament, part of a meniscus, labrum, etc. The problem with injecting the ACL to repair partial tears, complete non-retracted tears, and complete retracted tears is that targeted delivery cannot be accurately accessed blind (without imaging guidance) because it's buried deep in the knee. In addition, commonly used ultrasound imaging can't see tissues well enough to make injections using musculoskeletal ultrasound practical. The utilization of C-arm and high definition ultrasound guidance into joints has grown in regenerative medicine, however application of a small arthroscope to directly visualize the pathology being treated and cell therapies being applied has been proven to be far superior to other imaging modalities.

### Cell Delivery to Spine Targets

Inserting cells into the spine intrathecally, (meaning inside the covering of the spinal cord), typically requires imaging to make sure cells are really getting to the target. Without direct visualization, the physician may or may not be in the desired space. Being a few millimeters too shallow or too deep can mean not getting cells to the injured area. Even if at the desired space, the physician has no way of knowing if the cells will ever make it to the injury site, because he has no way of observing if cells will travel up, down, right, or left. Cells that are delivered may all get stuck in a spot which is nowhere near the lesion. If the injection of stem cells had used direct visualization, observations could be made to determine if cells are likely or not likely to make it to the injury site. Obviously, if the cells can't make it to the area in need of repair, injecting stem cells may do no good. The cell delivery catheter system and method of use will insure injection of cells in the right spot. Please note, that ACL and spine procedures are not all inclusive of the types of procedures that may utilize the catheter system. Clearly there is a need for such a device because most physicians currently use devices intended for other uses and not specifically for cell delivery. When using the catheter system described, patients are exposed to a more predictable and precise delivery; reducing the risk of procedure failure and increasing the probability of successful treatment.

### BRIEF DESCRIPTION OF THE INVENTION

The purpose of the present invention is to provide an arthroscopic lavage system that permits examination, diagnosis and treatment through a small single-port of entry, allowing a physician to use a minimally invasive, direct visualization approach which is more accurate and a superior diagnostic modality compared to X-ray, MRI and high definition ultrasound.

Suction and irrigation have been standard features in operative arthroscopy (joint), laparoscopy (abdomen/pelvis), cystoscopy (bladder), and hysteroscopy (uterus) for many years. Laparoscopy and hysteroscopy currently use carbon dioxide (CO2) gas as the primary distension media which is electrically monitored and controlled by an insufflator. Hysteroscopy also uses fluid as the distension media, similar to operative arthroscopy and cystoscopy. Operative arthroscopy, hysteroscopy, and cystoscopy primarily use irrigation fluid as distension media only, allowing the specific cavity or organ to be extended or open for viewing and performing operative tasks.

The suction and irrigation aspect in laparoscopy uses saline fluid in a lavage fashion for flushing and cleaning the cavity/tissue/organ of blood and debris for better visualization, but the distension of the abdomen is accomplished with CO2. Both arthroscopy and laparoscopy require a sharp puncture through tissue to enter the respective cavity while in hysteroscopy, the entry into the uterus is through the vagina and dilation of the cervix, and in cystoscopy through dilation of the urethra. No sharp instruments are used or required in either for the purpose of cavity entry.

The primary function of the ‘wet’ inflow fluid used in operative arthroscopy is for distension and generally has inflow entering through one port and outflow through a second port or through the shaving or other device introduced through a second port. The sheath that an operative arthroscope is inserted through is generally more than twice the size of the 4-5 mm arthroscope. Hysteroscopy and cystoscopy also utilize an outer sheath around the scope which allows for both the constant inflow and outflow of fluid, preset at specific volume and flow levels and controlled by machine. When fluid is the primary distension media in hysteroscopy, flow can also be controlled intermittently with the use of a physician operated foot pedal.

The arthroscopic lavage system of the present invention is distinguished from the usual system and method described above because the primary function of the irrigation/inflow fluid is for cleaning and its' secondary function is that of distension. Both directed inflow and outflow are intermittent and totally physician controlled by trumpet valve buttons on a handpiece. In laparoscopy irrigation fluid inflow and outflow are intermittently controlled via trumpet valve buttons similar to the system disclosed herein, but the suction/irrigation is accomplished through a separate suction/irrigation device introduced through a second, separate port.

The device disclosed herein is a physician controlled suction/irrigation device that incorporates separate push button valves for both suction and irrigation, and also function as an entry cannula. It is also the only suction/irrigation device that doubles as the scope cannula and permits a single puncture only.

The arthroscopic lavage system is particularly adaptable to performing office/clinic and ambulatory surgery center based procedures. The single port entry system allows the physician to use minimally invasive direct visualization for diagnosis and also provide therapeutic benefit by completely cleaning and flushing the joint with sterile saline (lavage).

The single-port entry is facilitated through a unique disposable suction/irrigation handpiece and introducer set which permits easy cannula entry into the joint and also functions as the housing for the small arthroscope during the procedure. Separate pressurized irrigation and vacuum suction capabilities are incorporated in the handpiece for ease of use and is physician accessed on demand through finger-controlled trumpet valves. The disposable handpiece's integral suction and irrigation tubing set connects the handpiece to the respective dual canister vacuum pump and dual irrigation pump which is pressure controlled via a separate air compressor. These components are mounted on a portable procedure cart which also contains a video system housing the camera, light source, optical coupler and focus control. The video system also includes a high-resolution monitor for viewing and a video recording device for documenting the procedure, including the ability to digitally store and make video prints.

For removal of large loose bodies or the need for larger diameter instruments, devices and/or catheters being inserted through an auxiliary valve, the swivel cannula can be exchanged for a larger diameter ‘loose body’ removal/biopsy cannula. To make the cannula exchange without having to reacquire the portal pathway through tissue, the scope is removed from the swivel cannula and an exchange rod is inserted and advanced through the cannula and inside the joint capsule.

Following removal of the swivel cannula over the exchange rod, the swivel cannula is unscrewed from the suction/irrigation handpiece and the larger diameter ‘loose body’ cannula is screwed on. The exchange rod remains inside the joint prior to insertion of the larger diameter cannula and a tapered dilator shaft is slipped over the exchange rod and inserted into the joint, gently expanding the portal opening to accommodate the larger diameter sized cannula. The tapered dilator shaft is removed and the larger cannula and attached suction/irrigation handpiece are then slipped back over the exchange rod and inserted into the joint, eliminating the time-consuming nuisance of finding the original joint entry path through tissue. The exchange rod may then be removed and replaced with the arthroscope. Larger instrument, device and catheter diameters may now be accommodated and inserted through a larger diameter auxiliary valve mounted on the body of the larger diameter cannula, which can now remove larger diameter loose bodies and perform tissue biopsy under direct visualization.

Additional devices for use through the auxiliary valve of the larger diameter cannula permit cutting and ablation of tissue.

Following completion of the lavage the system disclosed is capable of a rapid turnover from a ‘wet’ fluid inflow/outflow cleaning and distension media to that of a ‘dry’ CO2 gas distension media also capable of directed tissue site drying. Connecting a CO2 gas insufflator tubing line directly into the irrigation/inflow tubing line, in place of the fluid irrigation pump and air compressor, allows for the physician controlled trumpet valves on the handpiece to now control ‘dry’ gas inflow and outflow in an identical manner employed during the ‘wet’ saline lavage. An alternate means of CO2 conversion for distension and directed tissue drying utilizes both auxiliary valves on the cannula, with 1 auxiliary valve open and connected to the insufflator, and the other auxiliary valve closed to maintain distension, or partially open, to allow gas to escape while maintaining distension and directed tissue drying by continuous gas flow. Still another means of CO2 conversion for distension and directed tissue drying utilizes a configuration of 1 auxiliary valve open and connected to the insufflator for inflow, the other auxiliary valve closed, and continuing to use the suction valve on the handpiece to control the level of distension and flow rate for directed tissue drying.

Following completion of directed tissue site drying, the disclosed system small diameter grasping instrument or device, is used to transport a biocompatible tissue scaffold through an open auxiliary valve on the cannula body and through the central channel of the swivel cannula, beyond the distal tip of said cannula and advanced to the dried tissue site. The scaffold can then be manipulated and accurately affixed to the dried tissue site with the grasper while under direct visualization and prior to removal of said grasping device.

Following completion of directed tissue site drying and placement of a biocompatible tissue scaffold, the disclosed system small diameter ‘cell placement’ catheter is inserted and advanced through an open auxiliary valve on the cannula body, through the central channel of the cannula, beyond the distal tip of said cannula and advanced to the prepared dried tissue site with tissue scaffold affixed. The system ‘cell placement’ catheter includes a direction controlled tip enabling precise, accurate cell placement and layering of autologous regenerated cells to the scaffold.

An alternative method to placing cells on a tissue scaffold is to place the regenerated cells directly on a dried tissue site. Following directed tissue site drying as described above, the disclosed system small diameter ‘cell placement’ catheter is inserted and advanced through an open auxiliary valve on the cannula body, through the central channel of the cannula, beyond the distal tip of said cannula and advanced to the prepared dried tissue site.

The system ‘cell placement’ catheter enables precise, accurate cell placement and layering of autologous regenerated cells directly to a dried tissue site or sites within the joint while under direct visualization and through the same single, small port of entry.

A catheter was designed to utilize integrated elements and provides a relatively simple navigation mechanism to access multiple, topographically distinct injection sites for cell delivery. The catheter is steered by an operator using a control handle located at proximal end. The action of one or more pull wires embedded along the length of the catheter shaft creates tip deflection to allow access to target site. Catheter control handle mechanisms enables tip movement ranging from straight to semi-circle deflected positions. Under direct visualization, catheter is manipulated through a combination of axial rotation and deflection of the distal aspect. In its preferred embodiment, the catheter is introduced through the lumen of the system's single-port arthroscopic cell delivery device platform and steered to the desired target site for one or multiple treatments. This steerable cell delivery catheter allows for accurate placement of medications, platelets, or stem cells anywhere in the musculoskeletal system.

The catheter shaft is dimensioned with an outside diameter to allow catheter to be threaded through an endoscopic device for insertion into anatomic structures or spaces as necessary to perform a desired medical procedure. Catheter may also be introduced peripherally to access target sites via the human vasculature. An inside diameter is sufficient to accommodate cell/cell product delivery through a main lumen, steering wire(s) and/or balloon inflation channels; depending on the intended use of the catheter.

Shaft may be fabricated from Polyoxymethylene copolymer (POM), Polyamides, Polyetherimide, Polyetheretherketone, Polyethylene or other materials commonly used in the manufacture. All materials are biocompatible to minimize physical effects on cells during the delivery process. Hydrophilic coating on shaft may facilitate introduction of catheter beside other devices in lumen of arthroscopic device.

To accomplish steerability or deflection of shaft or tip, shaft construction may utilize an inner liner, braided wire layer and outer jacket. A single or multiple pull wires may be incorporated to enable steering for navigation. Numerous mechanical properties are advantaged to optimize functioning, i.e., the shaft resists compression during use and transmits torque. Operator is able to advance catheter shaft to target site, sometimes against significant frictional resistance, without undue axial compression or snaking of the shaft.

The wire braid layer may be composed of round, flat or coil wire. The pattern of braid is controlled to vary pitch, diameter, and tension to achieve required torque and pushability. Materials may be stainless steel, nitinol, platinum or iridium. In the preferred embodiment, the shaft is ˜2 Fr in size but may range from 1.9 Fr to 4 Fr.

Overall working length of catheter may range from 65 to 150 cm. Shaft may be filled with radiopaque material (barium sulfate, tungsten, etc) for imaging and contain printed elements such as incremental depth markings, etc. The distal tip may be open or closed and may contain a pull ring connected to pull wire(s) to enable steering of the catheter. The open tip may be of soft durometer and terminate in a tapered, radiused, necked or chamfered opening.

A closed tip design may be configured with port(s) proximal to tip to allow delivery of cell products.

A control handle is connected to proximal end of catheter shaft and may be constructed with the following elements; 1) cell delivery lumen that terminates proximally in a luer lock connector for attachment of a syringe 2) one or more pull wire(s) proximally connected to control knob(s). Distal end of pull wire(s) terminates distally in shaft tip or a pull ring. Movement of control knob(s) results in movement of catheter body or tip via pull wire(s). 3) luer stopcock(s) are connected to proximal inflation lumen to enable inflation of one or more balloons positioned at distal end of shaft.

Single or dual balloon configurations may be round, elongated or bell shaped; depending on procedure and target site. The inflated external diameter range is 2 to 25 mm. A bell shaped balloon may be fabricated to direct cell products to target site. Typical balloon wall thickness can range from 0.01 to 0.2 mm. Overall working length is 10 mm to 150 mm. Construction materials may include urethane, nylon, PTFE or PET. Bioactive material may be applied to coat outer surface of the expandable balloon, i.e., an anti-inflammatory steroid, anti-microbial.

For vascular interventions, balloon(s) may be incorporated and deployed to prevent cell washout and increase the dwell time (the time during which the injected cells remain undisturbed by the resumption of blood flow). Occlusive balloon inflation is initiated just before injection of the cell suspension.

Cell delivery catheter is dimensioned for introduction through common lumen of single-port arthroscopic device, or can be peripherally inserted for vascular access. A steerable catheter allows physicians to target delivery and account for any peculiarities of a patient's anatomy, and self-locking “curves” remain in place throughout the procedure. The ergonomic handle facilitates tactile response while positioning catheter for ease of placement. Distal shaft configurations may include 1) a bell shaped balloon 2) dual balloons with port(s) located between balloons 3) open distal tip or 4) closed tip with proximal ports to enable precise placement of cells and/or products. A low durometer tip helps prevent trauma and offers superior tissue contact.

The small single-port arthroscopic lavage, directed tissue drying, biocompatible tissue scaffold placement and autologous regenerated cell placement delivery system of the present invention is intended as a diagnostic procedure for accurate joint evaluation while therapeutic lavage provides long-term (3+ years) pain relief.

The mechanism through which this is accomplished is the simple and thorough flushing out of loose bodies and chemical irritants commonly found in chronic osteoarthritis (OA) and rheumatoid arthritis (RA) afflicted joints. The small size of the devices (less than half the size of standard, operative arthroscope) and single-entry port rather than multiple large punctures and ports in standard operative arthroscopy, make this procedure ideal for a physician's office, clinic or ambulatory surgery center.

The procedure is performed under local anesthetic only and in conjunction with a mild oral sedative (e.g., Valium), eliminating the additional risks and associated complications of general anesthesia. An alternative to local anesthesia is a spinal epidural injection, predicated on physician preference. Patients undergoing the procedure experience minimal discomfort and return to normal activities within 24-48 hours.

Use, accurate placement and the adherence of regenerated cells to a prepared site or sites within a joint combines the documented, immediate pain relief of joint lavage with the stimulation, repair and re-growth of damaged tissue and structures within the joint to promote natural tissue and joint regeneration. From the patient's perspective, natural joint regeneration is far superior to the accepted alternative of highly invasive major surgery and total or partial joint replacement.

At the onset of the procedure, following administration of local anesthesia and a 5 mm incision, a sharp trocar is inserted into the suction/irrigation handpiece and attached swivel cannula. The working end or cannula tube is then inserted into the joint to the level of the joint capsule. After piercing the surface tissue the sharp trocar is replaced with a blunt obturator and “popped” into the interior of the joint through the joint capsule. The blunt obturator is removed and replaced with the arthroscope and after attaching the disposable handpiece integral suction/irrigation tubing set to the respective devices located off the sterile field; irrigation and aspiration of the joint with sterile saline commences while simultaneous evaluation and diagnosis is accomplished under direct visualization.

Alternately irrigating and suctioning until a clear picture is obtained, the diagnosis is performed while continuing to flush as needed to maintain a clear operative field and to thoroughly wash out the loose bodies and irritants contained within the joint. Generally 1 to 3 liters of normal sterile saline are used to perform the lavage and to cleanse the joint completely of loose debris and degradative enzyme contaminated synovial fluid. A larger diameter cannula for removal of large loose bodies or when larger instrumentation, devices and/or catheters are needed, the procedure for exchanging the swivel and larger diameter biopsy cannula can be used. The swivel and larger diameter loose body/biopsy cannula are attached to a threaded coupling or fixture that includes auxiliary stopcocks or ball valves that allow for removal of sterile synovial fluid and loose bodies for laboratory analysis, and also permits direct injection of anesthetic, drugs and regenerated cells into the joint.

In addition, cannula stopcock and/or ball valves are also used for insertion and removal of system instruments, devices and/or catheters. The invention disclosed herein is the only application of an additional valve or valves on the attached cannula and suction/irrigation device which is different from the large number of standard entry trocar/cannula that utilize a valve for distention purposes only.

The system described also includes the use of video coupling optics connected to the camera head, camera control module and combination light source in a single enclosure located off the sterile field.

This eliminates the need to have a camera head and cable, optical coupler, light cable and scope all sterilized and then assembled on the field.

The system design also eliminates the weight of the camera head and cable, optical coupler and light cable from creating an unbalanced design weighting down a very lightweight scope, cannula and handpiece configuration. The only video train component in this system disclosed herein needing sterilization is the arthroscope which contains integral illumination fibers, allowing elimination of a separate light cable. The 1.7 mm scope in this system uses a 30,000 pixel fiber image bundle with a two-element distal lens which provides excellent image quality, large field of view, and depth of field approaching that of a 4 mm rod lens arthroscope.

Immediately following completion of the joint lavage, the integral inflow/irrigation tubing attached to the handpiece is disconnected from the irrigation pump and sterile saline fluid container. The system described herein also includes the use of a CO2 gas insufflator and tubing which is then attached or coupled directly into the integral inflow/irrigation tubing by means of a ‘Christmas tree’ or stepped connector. The inflow/irrigation tube has remained attached to the handpiece and controlled by the inflow/irrigation trumpet valve located on the handpiece. When the inflow/irrigation trumpet valve is now depressed, the flow of CO2 gas replaces saline as the distension media, and directed tissue drying of a specific site can commence. The suction valve on the handpiece continues to be used as previously described.

Following directed tissue drying the advancement of the system grasping instrument or device to place a biocompatible tissue matrix is accomplished under direct visualization as previously described.

The system of the invention described herein immediately follows directed tissue drying with the advancement of the system cell delivery catheter to place autologous regenerated cells accurately and precisely onto a dried tissue site or sites as previously described; or immediately following the placement of a securely affixed biocompatible tissue scaffold to a dried tissue site or sites, the advancement of the system cell delivery catheter to place autologous regenerated cells accurately and precisely directly to a biocompatible tissue scaffold site or sites as previously described.

The system of the present invention provides an all-inclusive direct visualization, single-port arthroscopic lavage, tissue drying, biocompatible tissue scaffold placement, and autologous regenerated cell placement delivery system. The intent of the present invention is to allow a physician to treat OA, RA and other destructive joint diseases while using a minimally invasive/least invasive approach that eliminates the added risk of general anesthesia and highly invasive major surgery/joint replacement. The system provides the ability to thoroughly and efficiently clean the joint by means of a pressurized inflow/irrigation circuit and vacuum outflow/suction circuit.

The system easily and rapidly maintains distension following lavage, and provides the ability for directed tissue site drying, by converting to CO2 gas inflow regulated and flow controlled by the system insufflator and inflow/irrigation trumpet valve on the handpiece. The present invention also allows the physician to use the system grasping instrument or device to place and affix a biocompatible tissue scaffold to a dried tissue site or sites within a joint; and the present invention also permits use of the system catheter for precise, accurate placement and layering of cells onto both a dried tissue site and/or a previously affixed biocompatible tissue scaffold.

The intention of the disclosed system invention is to provide patients with significant, immediate long lasting pain relief by way of lavage, and in also providing a cleansed joint for superior patient outcomes when implanting autologous regenerated cells to dried tissue sites or affixed biocompatible tissue scaffolds to stimulate and promote tissue growth and natural joint regeneration. The disclosed system invention provides a means for the immediate elimination or significant reduction of pain and creates an ideal, clean environment for the improved success of regenerated cells to promote new tissue growth. This new treatment modality and protocol provides a return to improved long-term joint health and function, while eliminating the pain, general anesthesia, hospital borne infection and complication risks, and the long-term rehabilitation and physical therapy necessary in major invasive surgery and joint replacement. Additionally, the disclosed system invention and method represents a significant reduction in cost to the healthcare system when compared to the current, approved treatment protocols of total and partial joint replacement.

Other objects, advantages, and novel features of the invention will become apparent from the following detailed description when considered in conjunction with the accompanying drawings where in like reference numbers and identifying light parts throughout.

### BRIEF DESCRIPTION OF THE DRAWINGS

FIG. 1 is a plan view of a handpiece for use in arthroscopic lavage, tissue drying, biocompatible tissue scaffold and autologous regenerated cell placement delivery system illustrating the connection of the dual valve swivel cannula and rear introducer/entry set and scope lock adaptor.

FIG. 2 is a plan view illustrating the insertion of a sharp trocar through a disposable handpiece and dual valve swivel cannula for piercing the entry site tissue directly above the joint.

FIG. 3 is a plan view illustrating the insertion of the handpiece with trocar through the entry site tissue of the knee joint with the sharp trocar locked in place and penetrating the tissue to the depth of the joint capsule.

FIG. 4 is a partial sectional view taken at 4-4 illustrating the sharp trocar locked in place in the swivel cannula piercing the entry site tissue.

FIG. 5 is a plan view illustrating the replacement of the sharp trocar with the blunt trocar (obturator) for puncturing the joint capsule.

FIG. 6 illustrates the insertion and locking in place of the obturator through the dual valve swivel cannula.

FIG. 7 is a partial sectional view taken at 7-7 illustrating the obturator in the dual valve cannula penetrating the joint capsule and inside the interior joint space.

FIG. 8 is a plan view of the arthroscopic lavage system disposable handpiece with the dual valve swivel cannula attached illustrating insertion of the arthroscope.

FIG. 9 illustrates the dual valve swivel cannula in the knee joint with the arthroscope locked in position.

FIG. 10 is a sectional view taken at 10-10 of FIG. 9 illustrating the dual valve swivel cannula in the joint and the arthroscope locked in place.

FIG. 11 is a sectional view taken at 11-11 illustrating the distal, circumferential structure of the dual valve swivel cannula and arthroscope. The distal optics of the arthroscope are also illustrated.

FIG. 12(A) is a combination top/side view showing the orientation of the irrigation and suction valves and integral tubing.

FIG. 12(B) is a cross-sectional view of the disposable handpiece of FIG. 12(A) illustrating the operation of the irrigation and suction valves and their respective flow channels.

FIG. 13 is a plan view illustrating the insertion of an exchange rod for removing and exchanging the dual valve swivel cannula.

FIG. 14 illustrates the removal of the dual valve swivel cannula and replacement with the dilator shaft using the exchange rod.

FIG. 15 is a plan view showing the tapered dilator shaft over the exchange rod and inserted in the joint.

FIG. 16 illustrates removal of the tapered dilator shaft and placement of the larger diameter ‘loose body’ removal and ‘biopsy’ cannula over the exchange rod and into the joint.

FIG. 17 illustrates the removal of the exchange rod after placement of the biopsy cannula into the joint.

FIG. 18 illustrates placement of the arthroscope into the biopsy cannula.

FIG. 19 illustrates the insertion of the biopsy instrument through the auxiliary valve on the biopsy cannula.

FIG. 19A illustrates a variation of the optional embodiment of FIG. 19 that allows insertion of a biopsy instrument through an auxiliary valve at the rear of a cannula body and eliminates the requirement for an exchange to a second, larger diameter cannula.

FIG. 19B illustrates an optional arrangement for insertion of a biopsy instrument through an auxiliary valve on the rear of the handpiece.

FIG. 19C illustrates a variation of the optional embodiment of FIG. 19B.

FIG. 20 is a sectional view illustrating the biopsy cannula with biopsy instrument and arthroscope in place inside the joint capsule.

FIG. 21 illustrates the arthroscopic lavage, tissue drying, tissue scaffold and regenerated cell delivery system procedure cart housing the video monitor, camera, light source, optical coupler and focus mechanism, video recording device, air compressor and dual irrigation pump, suction unit and dual collection canisters with suction and irrigation tubing attached and proximal arthroscope inserted into the separate optical and illumination receptacles on the front of the video system.

FIG. 22 illustrates the arthroscopic lavage, tissue drying, tissue scaffold and regenerated cell delivery system procedure cart housing the insufflator, turning off the air compressor and dual irrigation pumps, and the connection of the insufflator tubing into the irrigation/inflow tubing to maintain distension by means of the CO2 insufflator, and to also provide directed tissue drying.

FIG. 23 illustrates the dual auxiliary valves on the swivel cannula in a configuration allowing continual gas flow for tissue drying while 1 valve is attached to the insufflator and the other valve remains fully or partially open.

FIG. 24 illustrates the dual auxiliary valves on the swivel cannula in a configuration allowing distension only with 1 valve open and attached to the insufflator and the other valve remains closed.

FIG. 25 illustrates the dual auxiliary valves on the swivel cannula in a configuration allowing distension with 1 valve open and attached to the insufflator and the other valve also open allowing the insertion of the system grasping device for placement of a tissue scaffold.

FIG. 26 illustrates the dual auxiliary valves on the swivel cannula in a configuration where distension is maintained and controlled using the trumpet valves on the handpiece with 1 auxiliary valve open allowing the insertion of the system grasping device for placement of a tissue scaffold.

FIG. 27 illustrates a sectional view of the cannula with grasping device and arthroscope in place inside the joint capsule.

FIG. 28 illustrates the dual auxiliary valves on the swivel cannula in a configuration where distension is maintained and controlled using the trumpet valves on the handpiece with 1 auxiliary valve closed, and the other auxiliary valve open allowing the insertion of the system cell delivery catheter.

FIG. 29 illustrates a sectional view of the cannula, arthroscope and cell delivery catheter inside the joint capsule, with catheter depicting various stages of tip deflection and stabilizing balloon inflation.

FIG. 30 illustrates the cell placement catheter device and control handle.

FIG. 30A illustrates a close up of the cell placement catheter device and control handle.

FIG. 30B illustrates the cut away view of the construction of cell placement catheter shaft.

FIG. 30C illustrates a cross section of the cell placement catheter control handle.

FIG. 30D illustrates a cell delivery catheter inserted into and through the cannula. The control handle with an inflation luer for inflation of the isolation/stabilizing balloon, depicted inflated at the catheter tip.

FIG. 30E illustrates close ups of the cell delivery catheter with the deflated balloon at the catheter tip, the inflated balloon at the catheter tip, and the inflated balloon with catheter end deflected for precise cell delivery.

FIG. 30F illustrates a cell delivery catheter and control handle with 2 inflation luers for inflation of 2 stabilization/isolation balloons, 1 stabilization balloon located at the tip of the catheter and 1 isolation balloon a specified distance from the catheter tip.

FIG. 30G illustrates a close up of the cell delivery catheter with both the stabilization and isolation balloons inflated, and also with both balloons deflated.

FIG. 30H illustrates as a reference only, examples of non-proprietary extrusions

FIG. 30I illustrates as a reference only, the relative size of the catheter device control handle.

### DETAILED DESCRIPTION OF THE INVENTION

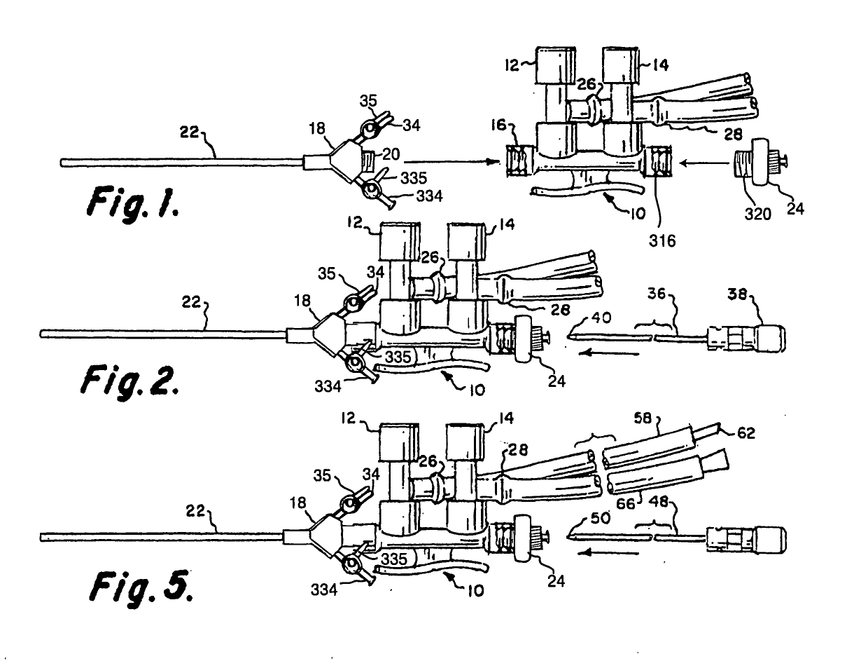

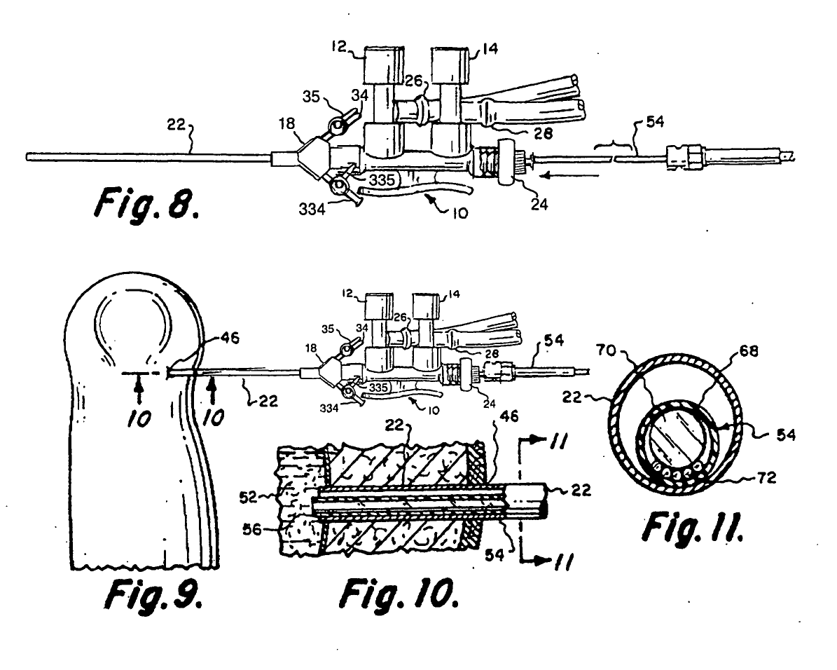

The arthroscopic lavage and tissue drying procedure handpiece components of the system are illustrated in FIG. 1 and are comprised of a handpiece 10 having trumpet valves 12 and 14 and threaded socket 16 for receiving coupling 18 having threaded nipple 20 and a dual valve cannula 22. Threaded nipple 20 is threaded into socket 16 in handpiece 10 to secure dual valve cannula 22 to handpiece 10. Fitting 24 on the opposite end of handpiece 10 has threaded nipple 320 and socket 316 for receiving coupling 24 to receive instruments to pass through handpiece 10, coupling 18 and dual valve swivel cannula 22 as shown in FIGS. 2 through 10.

Handpiece 10 also has irrigation and suction tubes 58 and 66 attached to trumpet valves 12 and 14 through channels 26 and 28. A unique feature of the invention is the inclusion of 2 auxiliary stopcock or ball valves 34 and 334 attached to coupling 18 which may be used for direct medication or cell placement into the joint and/or sterile synovial fluid removal. Auxiliary valves 34 and 334 are also to control distension and directed tissue drying when using a CO2 insufflator, and also for the insertion of the system grasping device for placement of a biocompatible tissue scaffold, and for insertion of the system cell placement catheter to deliver autologous regenerated cells to a specific tissue site within a joint. CO2 distension and tissue drying, placement of tissue scaffolds, and placement of regenerated cells will be described in greater detail hereinafter.

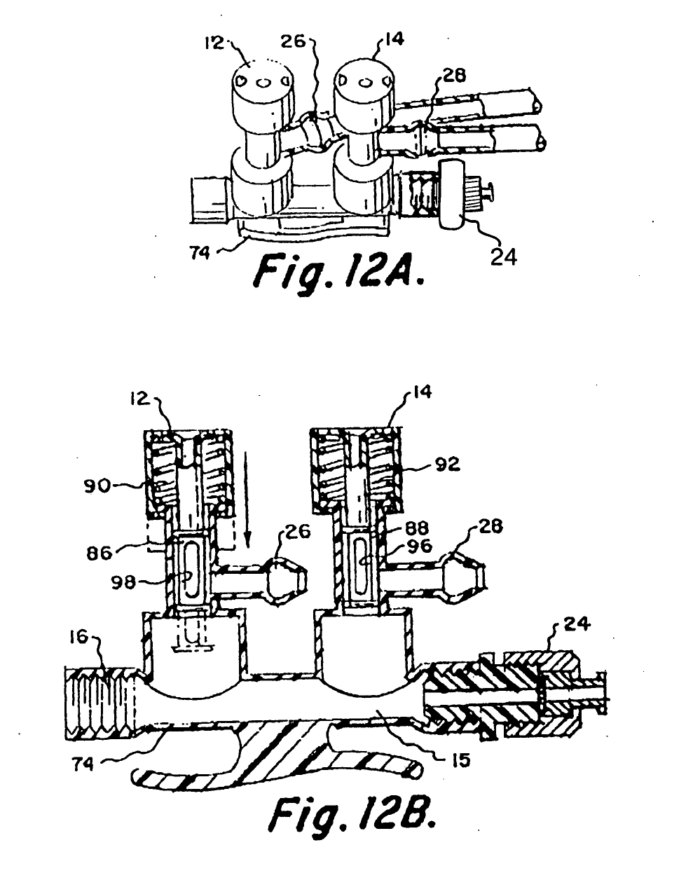

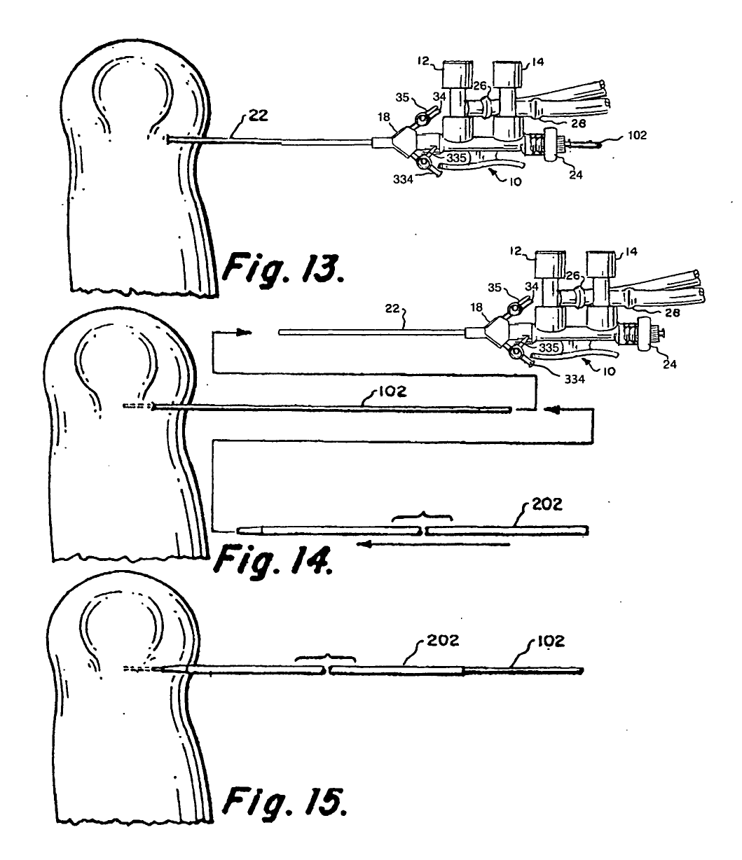

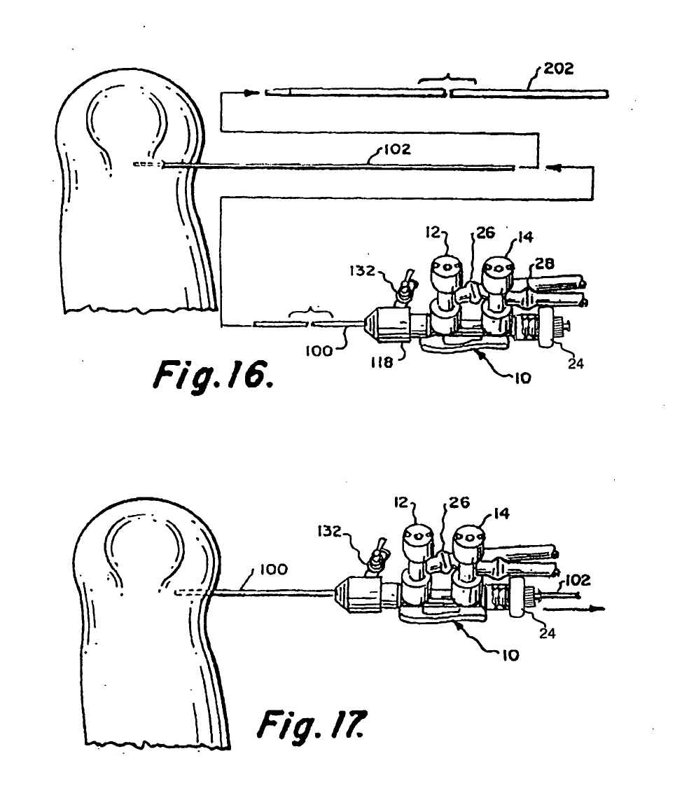

The placement of the dual valve cannula of the system is illustrated in FIGS. 2 through 7. Initially an introducer in the form of a sharp trocar 36 having handle 38 is inserted into fitting 24 in handpiece 10, passed through channel 15 (FIG. 12B) and passed through cannula body 18 until the sharp tip 40 extends out of dual valve cannula 22 as illustrated in FIG. 4. Sharp trocar 36 is used to pierce the skin 42 and surface tissue directly above the joint at the point of insertion until it reaches the joint capsule 44, creating entry portal 46.

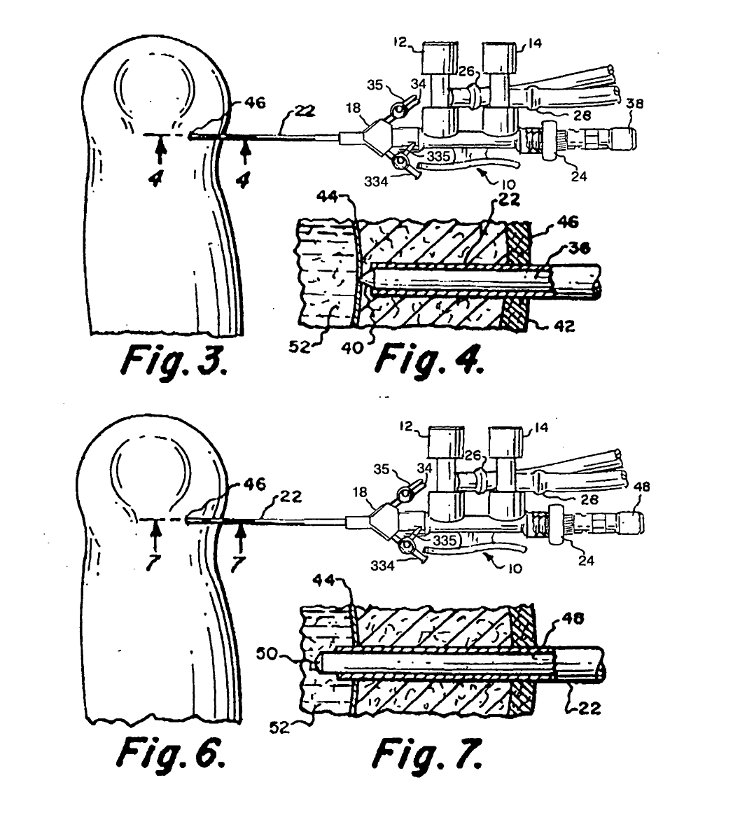

FIG. 3 illustrates placement of the arthroscopic lavage, tissue drying, tissue scaffold and regenerative cell placement system handheld device in a knee joint but, of course, the system may be used for other joints as well. After dual valve cannula 22 with sharp trocar 36 reaches joint capsule 44, sharp trocar 36 is withdrawn and replaced with blunt obturator 48 having a blunt end 50. Blunt obturator 48 is passed through fitting 24 in handpiece 10 and dual valve cannula 22 as shown in FIGS. 6 and 7. With dual valve cannula 22 in portal 46, blunt obturator 48 is pushed (i.e. “popped”) through joint capsule 44 into interior joint space 52. Dual valve cannula 22 is now positioned in interior joint space 52 ready for use in examining the joint.

An arthroscope 54 is then inserted through and locked into fitting 24 in handpiece 10 into dual valve cannula 22 as illustrated in FIG. 10. The entire working length of dual valve cannula 22 may now be extended through portal 46 deep into joint compartment 52 with distal end 56 of fiberoptic arthroscope 54 inserted to view and inspect all compartments such as the superpatella pouch, patellofemoral joint space, medial recess, medial compartment, intercondylar notch, lateral compartment, and lateral recess. During the inspection and examination, the joint is distended by irrigation solution inflow through tube 58 (FIG. 5). The irrigation tube 58 has spike 62 for puncturing a seal on irrigation solution (sterile saline) bags (not shown). Irrigation solution is released through irrigation and suction handpiece 10 by operation of trumpet valve 12. The irrigation fluid distends joint space 52 allowing visualization of the interior of the joint.

Direct insertion of medication into the interior joint space and/or removal of sterile synovial fluid may be performed through auxiliary ball valve 34. Medication is inserted by opening auxiliary ball valve 34 by rotating handle 35 or through auxiliary ball valve 334 by rotating handle 335, providing an entry/exit path through fitting or coupling 18 into dual valve cannula 22.

Medication can then be injected through ball valve 34 or valve 334 into the interior joint space 52. Alternatively, an empty, sterile syringe can be attached to the end of ball valve 34 or 334 for removal of sterile synovial fluid for analysis.

The single-port arthroscopic procedure is performed by visualizing the interior joint space 52 through arthroscope 54 while irrigating the joint cavity with sterile saline solution through irrigation tubing 58, connected through irrigation channel 26 by operating trumpet valve 12 to distend the joint. This fills and distends the joint allowing visualization of interior joint space 52 through fiberoptic arthroscope 54.

Distal end 56 of arthroscope 54, locked in position in handpiece 10, may be manipulated by moving handpiece 10 around to visualize the inside of interior joint space 52.

After irrigation and distention, suction may be applied by operating trumpet valve 14, through suction channel 28 connected to suction tube 66 (FIG. 5) flowing to dual suction collection canisters as will be described in greater detail hereinafter. The irrigation and suction system is used to remove loose bodies, debris and other irritants contained within a diseased joints interior joint space 52. Removal of loose bodies, debris and irritants is found to be beneficial in significantly reducing pain and increasing mobility, particularly to those suffering from osteoarthritis.

Arthroscope 54 is a small (approximately 1.7 mm) stainless steel sheath 68 (FIG. 11) preferably containing a 30,000 pixel fiberoptic image bundle and having a distal glass lens 70 for viewing the interior of joint space 52 with a CCD, CMOS or other type of photo sensor camera. Illumination fibers 72 contained in sheath 68 are provided for illuminating joint space 52 with high-intensity light. The outside diameter of fiberoptic arthroscope 54 is approximately ½ the inside diameter of dual valve cannula 22. This allows larger pieces of cartilage or debris in joint space 52 to be suctioned out through cannula 22 without removing fiberoptic arthroscope 54. The details of the irrigation, suctioning, and manipulating handpiece 10 are illustrated in FIGS. 12(A) and 12(B). FIG. 12(A) is a top/side view illustrating the orientation of trumpet valves 12 and 14 in handpiece 10. Trumpet valves 12 and 14 are in line, with channels 26 and 28, and tubes 58 and 66 slightly offset from each other. This ergonomic arrangement permits handpiece 10 to fit comfortably in the hand of a physician with the index and middle fingers conveniently resting on trumpet valves 12 and 14.

As shown in FIGS. 12(A) and 12(B), irrigation and suctioning handpiece 10 is comprised of a main housing 74, preferably made of molded plastic or aluminum, having an interior passageway 15 connecting fitting 24 with threaded socket 16.

Irrigation and suction channels 26 and 28 respectively (FIGS. 12(A), (B)) are connected directly to trumpet valves 12 and 14 to control the flow of irrigation and suctioned fluids through dual valve cannula 22.

Trumpet valves 12 and 14 are comprised of stems 86 and 88 biased by springs 90 and 92 located at the top of each valve, into a normally closed position. Pressing down on either of trumpet valves 12 and 14 connects passageway 15 through valve stems 86 or 88 to either of channels 26 and 28. This construction allows the physician to manipulate dual valve cannula 22 by moving handpiece 10 around and injecting irrigating saline into, or suctioning fluid from joint space 52 as desired.

Irrigating fluid is supplied by pressing trumpet valve 12 to connect interior passageway 98 in valve stem 86 to channel 26. This allows irrigating fluid to flow from irrigating channel 26 into dual valve cannula 22 through main passageway 15.

Suction is provided in the same manner with irrigation trumpet valve 12 in the up or closed position.

When suction trumpet valve 14 is depressed, channel 28 is connected through passageway 96 in valve stem 88 to main passageway 15. This allows material to be suctioned from joint space 52 through handpiece 10 to collecting canisters as will be described in greater detail hereinafter. Thus the unique construction of irrigation and suction handpiece 10 allows the physician to visualize the interior of joint space 52 while irrigating and suctioning alternately as desired.

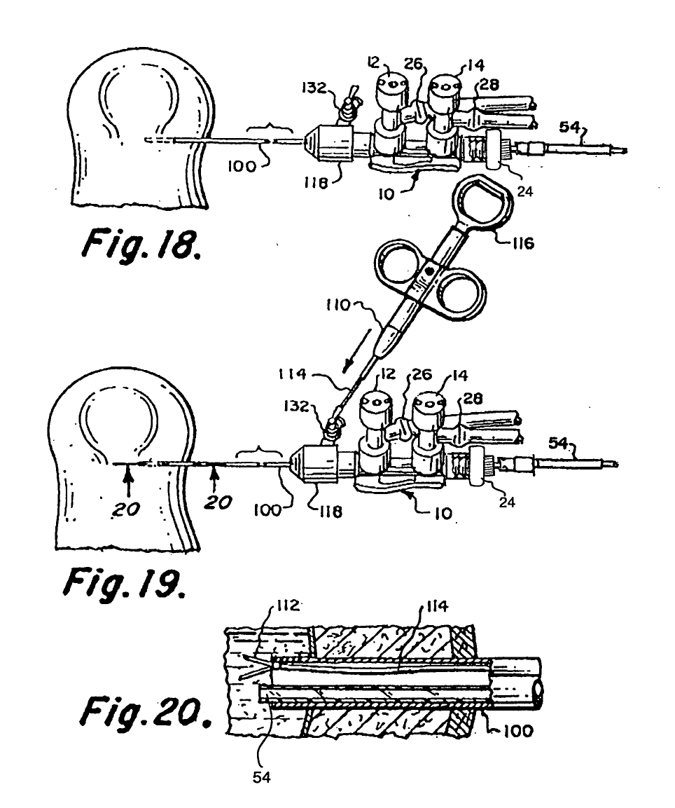

Another unique aspect of the invention is the ability to remove large loose bodies through irrigating and suctioning handpiece 10, and utilize larger diameter devices by exchanging the dual valve cannula 22 for a larger diameter cannula 100 as shown in FIGS. 13 through 16. This procedure is facilitated by use of an exchange rod 102 that is passed down through fitting 24 on handpiece 10 though dual valve cannula 22 until it is inside knee joint space 52. Dual valve cannula 22 may then be withdrawn with handpiece 10 as shown in FIG. 14. Dual valve cannula 22 may then be removed by detaching coupling 18 from handpiece 10 as illustrated in FIG. 1.

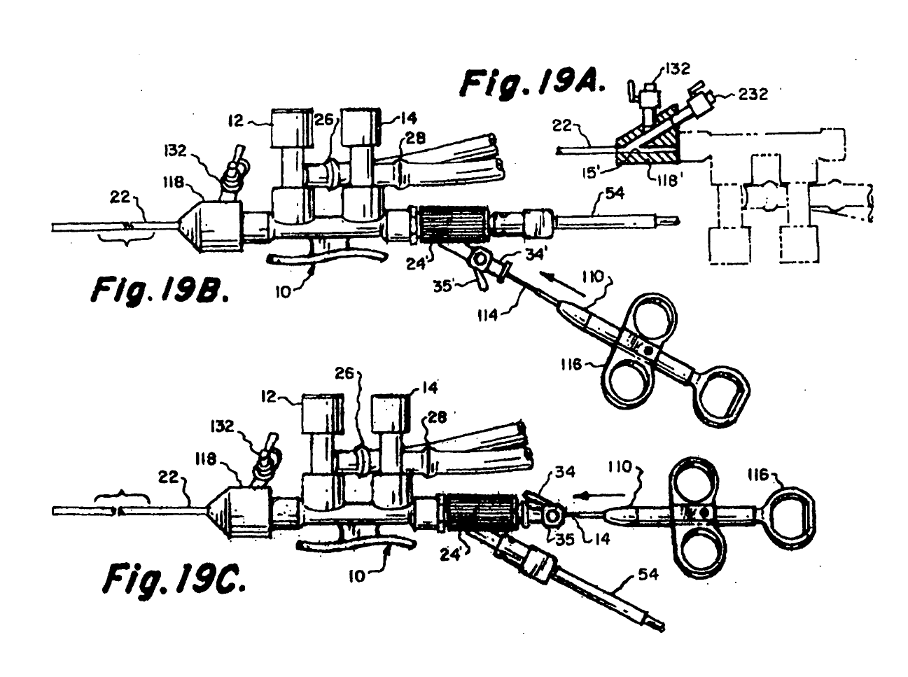

Larger diameter loose body cannula 100 is then attached to handpiece 10 by coupling 118 which also has integral stopcock or ball valve 132 for addition of internal joint medication with a syringe, removal of sterile synovial fluid if desired, or insertion of biopsy instrument 110. Tapered dilator shaft 202 is then fed over exchange rod 102 into joint space 52, gently expanding entry portal 46. Upon removal of tapered dilator shaft 102, large loose body cannula 100 is then fed over exchange rod 102 into joint space 52 and exchange rod 102 withdrawn as illustrated in FIG. 17 leaving larger diameter cannula 100 in place. Cannula 100 may now be used for performing removal of large loose bodies, and auxiliary valve 132 attached to cannula body 118 can be used to perform a biopsy or other procedures requiring larger diameter devices, under visualization, as illustrated in FIGS. 19 and 20.

With Loose Body cannula 100 in place, arthroscope 54 is inserted through fitting 24 of handpiece 10 into the joint space 52 as before.

As can be seen more clearly in FIG. 20, Loose Body cannula 100 is comprised of a larger central lumen and angled auxiliary valve 132. The lumen of cannula 100 allows visualization, suction, irrigation, removal of larger diameter loose bodies, tissue biopsy and procedures requiring larger diameter devices to be performed. Loose Body cannula stop cock valve 132 also receives a surgical device or instrument 110 such as biopsy forceps. Forceps jaws 112 at the distal end of flexible shaft 114 connected to surgical instrument 110 is operated by manipulating ring handle 116.

Forceps jaws 112 can be used to break up larger pieces of debris that might not fit through the larger diameter lumen of cannula 100 or can be used to cut, sample and remove tissue specimens from interior joint space 52. Loose Body cannula 100 is used to perform biopsies with biopsy forceps of approximately 2 mm in size being inserted through ball valve 132 allowing biopsies to be performed under direct visualization through arthroscope 54. Additional 2 mm devices for performing the tasks of cutting, shaving and ablation through ball valve 132 will also be available. The use of exchange rod 102 and tapered dilator shaft 202 eliminates the time-consuming nuisance of finding and gently enlarging original entry path 46 into interior joint space 52 adding an additional level of safety by eliminating the need to create a new entry path with the sharp trocar. While the system is described as performing an irrigation and lavage first and biopsy second, of course, the steps could be reversed or one used without the other. That is, the system can be used for a biopsy first followed by an irrigation and lavage or could be used to perform a biopsy or an irrigation and lavage separately, if desired.

The arthroscopic lavage, tissue drying, tissue scaffold and regenerated cell placement delivery system allows alternate irrigation and suctioning until a clear picture is obtained through the arthroscope and displayed on a monitor as will be described hereinafter.

While examination and diagnosis are performed, intermittent flushing is needed to maintain a clear operative field and to wash out loose bodies and irritants contained within the interior joint space 52. Generally up to 3 liters of sterile saline are used to perform the lavage and flush joint space 52. Should removal of larger loose bodies, a biopsy or use of another larger diameter device be desired, the procedure for exchanging dual valve cannula and loose body cannula as described hereinabove is employed.

Both inflow and outflow are intermittent and totally physician controlled via trumpet valve buttons 12 and 14 on irrigation and suctioning handpiece. The separate irrigation and suctioning capability incorporated in handpiece 10 are very efficient as they are physician accessed on demand through the finger control trumpet valves while manipulating handpiece 10.

In prior art devices and operative arthroscopy surgical procedures the inflow and outflow by irrigation and suction is preset with the primary function of distension being accomplished through separate inflow and outflow devices introduced through and requiring 2 separate ports. With the device disclosed herein, the suction and irrigation is both the distention media and also the primary therapy. The device is unique as it combines a separate physician controlled button valve for both irrigation and suction while doubling as an entry cannula and a scope cannula that permits a single puncture or port entry.

The use of the auxiliary stopcocks or ball valves on the front couplings of the dual valve and loose body cannula allow for removal of sterile synovial fluid and sterile loose bodies for laboratory analysis, as well as direct injection of anesthetic, medication and cells into the interior joint space 52, and also allows for instrument and device insertion, and specimen removal when using loose body cannula 100.

The inclusion of the auxiliary stopcocks and ball valves allows for the unique function of uninterrupted irrigation and suctioning on a larger scale, while also allowing the addition of anesthetics and medication, and also the removal of sterile synovial fluid and loose bodies on both cannula 22 and cannula 100, and biopsy specimens/tissue removal through valve 132 located on loose body cannula 100. In addition dual valve cannula 22 and auxiliary valves 34 and 334 can be used to control CO2 distension and CO2 flow rate during directed tissue drying which will be described in greater detail in a later section. Further, when open auxiliary valve 34 is connected to an insufflator, valve 334 can be used for insertion of the system grasping device and cell placement catheter while valve 34 continues to maintain CO2 distension. Reversing the connection of an insufflator to valve 334 and use of devices through valve 34 also allows for identical use and result. In the embodiment of FIG. 19, cannula body 100 is elongated by coupling 118 containing an angled or curved channel terminating in stopcock valve 132 that allows arthroscope 54 to stay in the joint and maintain its position while simultaneously inserting instrument 110 through auxiliary stopcock valve 132 and into the cannula central lumen when required. This embodiment is illustrated by stopcock valve 132 integrally formed on elongated fitting 118 which is part of the cannula body.

Optional embodiments for simultaneous instrument, device and catheter insertion while under direct visualization through a single port that eliminate the need for exchanging to a second, larger cannula are illustrated in FIGS. 19A, 19B, and 19C. In the embodiment of FIG. 19A, cannula body or fitting 118′ contains two separate stopcock valves communicating with internal channels 15′ and 115 contained within body 118′. Valve 132 is used exclusively for removal of sterile fluid and loose bodies, or infusion of medication through channel 115. Valve 232 at the rear of channel 115 is used for insertion and removal of instrument 110, and system grasping device and cell placement catheter eliminating the need to exchange cannula and also eliminating the need to remove an instrument or other device when intra-articular medication is desired.

This allows arthroscope 54 to stay in the joint while simultaneously inserting instrument 110 or other device through stopcock valve 232 and into the cannula central lumen when required.

Another optional embodiment for simultaneous instrument, device and catheter use under direct visualization through a single port, illustrated in FIG. 19B, also eliminates the need for exchanging cannula. An elongated “Y” or offset “V” type coupling or connector 24′ at the rear of disposable handpiece 10 allows arthroscope 54 to remain in the joint and maintain its position in one channel of the “Y” while instrument 110 is inserted through the other “Y” channel when required. In this embodiment valve 34′ has stopcock 35′ and receives instrument 110 through valve 34′.

Another alternative to the optional embodiment of FIG. 19B is illustrated in FIG. 19C. This embodiment also makes use of the “Y” or offset coupling on rear connector 24′ containing two channels.

However the coupling is constructed to allow the transposition of arthroscope 54 and instrument 110 illustrated in the embodiment of FIG. 19B. Embodiment 19C requires the working or distal end of arthroscope 54 to be flexible in order to traverse the angle of the offset scope coupling.

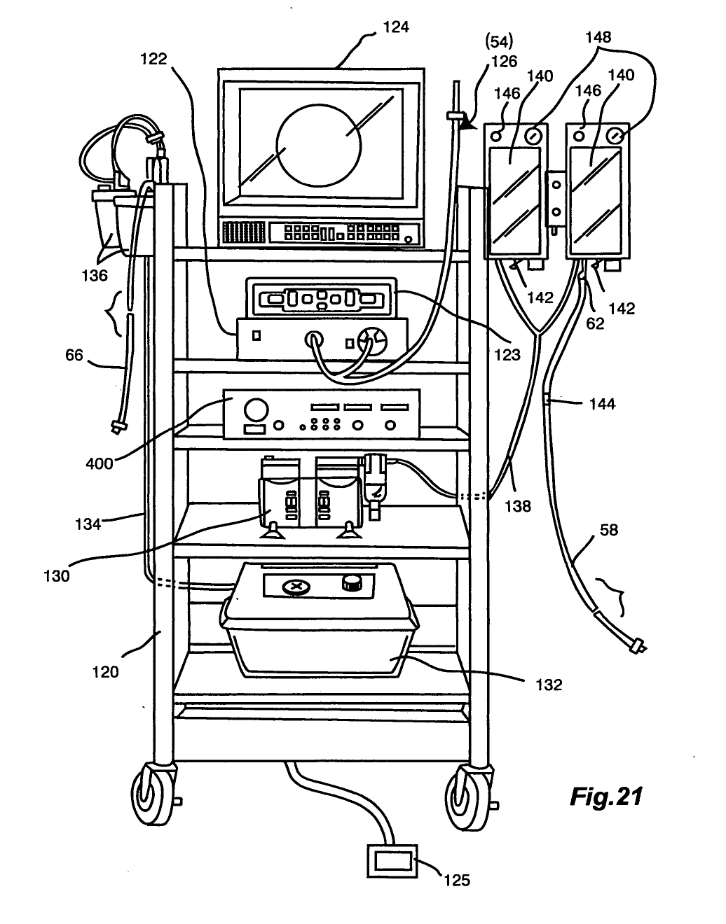

The entire operating system for visualization, pressurized irrigation & vacuum suction, tissue drying and documenting the procedure is mounted on a mobile self-contained cart for ease of movement and storage with a small footprint for use in a confined office/clinic or ASC environment. The support system for the arthroscopic lavage, tissue drying, and documentation is illustrated in FIG. 21. Portable cart 120 has shelves for receiving the components that work with the arthroscopic lavage system shown in FIGS. 1 through 20. A CCD camera, camera head, light source, optical coupling lens and focusing mechanism are all contained within console 122 which is mounted on the 2^{nd} shelf from the top of portable cart 120 along with digital image recorder 123. Recording device 123 is physician controlled by foot switch 125. High-resolution, flat panel monitor 124 is adjustable bracket mounted to cart 120 vertical supports. Console 122 is directly connected to arthroscope 54, 126 in FIG. 21, having distal lens 70 and light conducting fibers 72 as illustrated in FIG. 11.

Digital image recording device 123 also sits on the 2^{nd} shelf from the top of cart 120 and is used for documenting key aspects of the pathology and procedure.

Irrigation pressure is provided by air compressor 130, occupying the third shelf down, and controlled by irrigation pumps 140. Vacuum suction is controlled by console pump 132 located on the bottom shelf of cart 120. Vacuum suction console 132 is connected by vacuum hose 134 to collection canisters 136 mounted on an adjustable pole inserted in 1 of the 2 cart 120 vertical supports. Collection canisters 136 are connected to suction channel 28 on handpiece 10 by suction hose 66 as shown in FIG. 5.

Compressor 130 provides forced air through tubing 138 to dual irrigation pumps 140 each pressurizing 1 liter hanging bags of sterile saline. Sterile saline bags are connected to irrigation channel 26 on handpiece 10 by irrigation hose 58 with integral bag spike 62 (FIG. 5).

Irrigation pumps 140, also mounted on a second adjustable pole inserted in the other vertical support of cart 120, each having a switch 142 for selecting one or the other of the pumps to be pressurized. This allows an empty saline solution bag in one pump to be replaced while the flow is being delivered from the other pump. Separate pressure controls 146 and pressure gauges 148 are contained on pumps 140 for safe, accurate pressure control. Clamp 144 on irrigation hose 58 also closes to prevent premature flow of solution to suction irrigation handpiece 10. CO2 insufflator 400 is shown in FIG. 21 on the third shelf of cart 120, but is not utilized during the lavage portion of the procedure.

Following the lavage, a rapid conversion to CO2 distension and directed tissue drying is accomplished by turning off compressor 130 and irrigation pumps 140 by switching pump on/off switches 142 to the off position, and closing clamp 144 on irrigation tube 58.

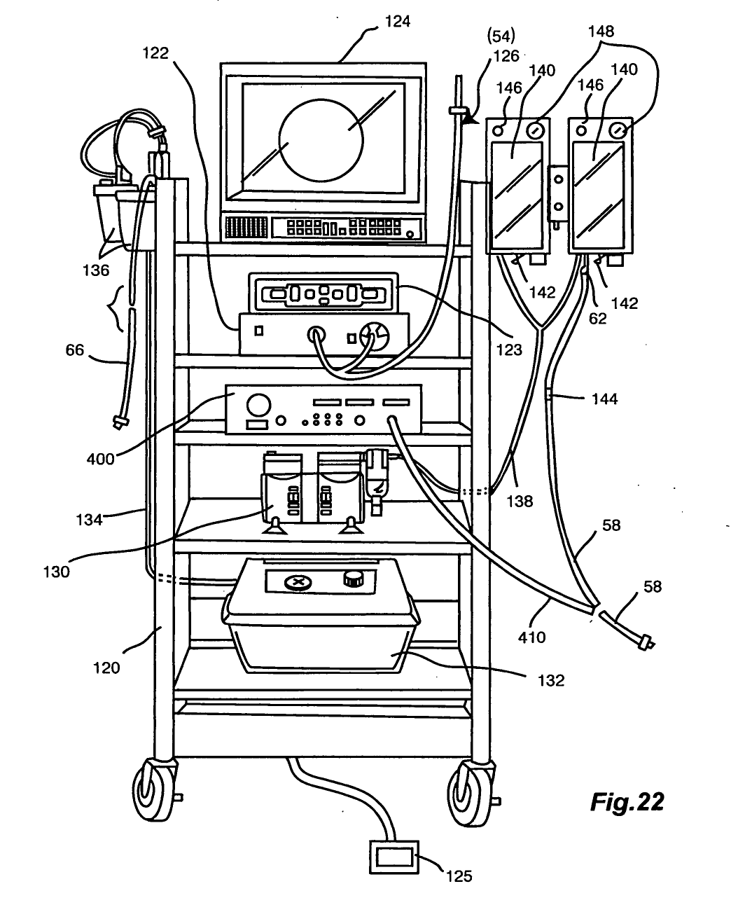

FIG. 22 illustrates insufflator 400 on cart 120 attached to insufflation tubing 410, which is attached directly to the severed end of inflow/irrigation tubing 58, after clearing the distal end of tubing 58 of irrigation fluid, by means of a standard stepped tubing adaptor (not shown). No changes to suction tubing 66 connections are required. In this preferred embodiment inflow of CO2 gas, distension and directed tissue drying are physician controlled by trumpet valves 12 and 14 on handpiece 10 in an identical manner to their use during lavage. Trumpet valve 12 remains attached to channel 26 which is still attached to the cut end of inflow/irrigation tubing 58, which is now attached to insufflation tubing 410 using a standard tubing connector. Depressing trumpet valve 12 now controls the inflow of CO2 gas, and depressing trumpet valve 14 which uses the same tubing configuration and connections as previously detailed, controls the outflow of CO2 gas.

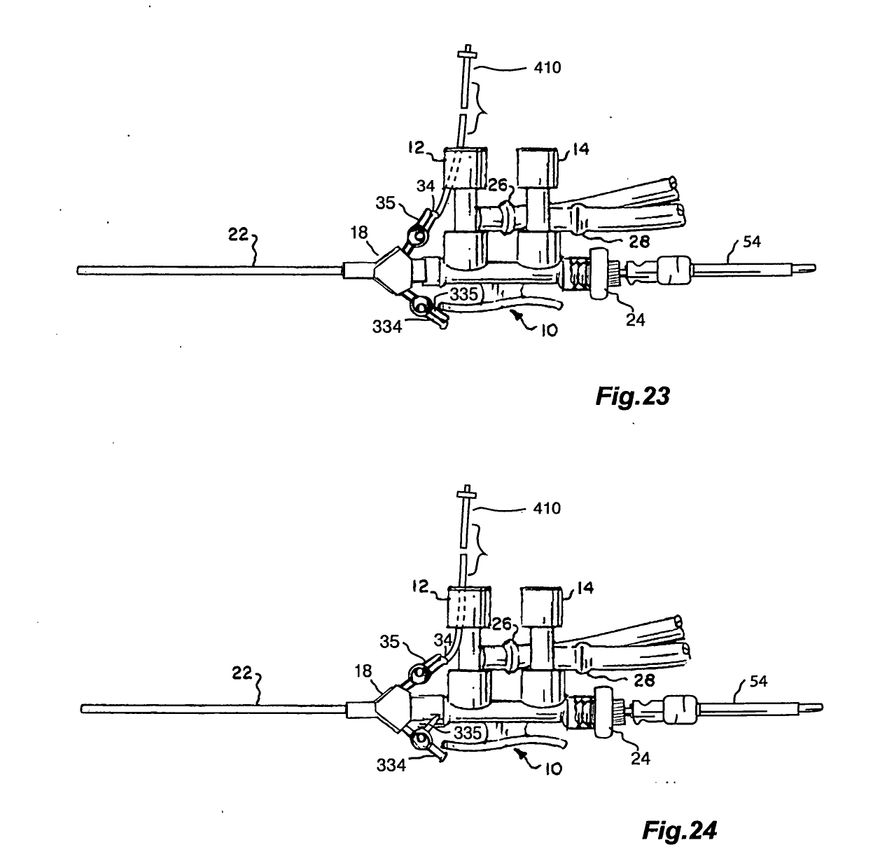

Another embodiment for rapid conversion to CO2 distension and directed tissue drying following lavage is illustrated in FIG. 23 and FIG. 24. In this embodiment the 2 auxiliary valves located on cannula body 18 are used to control the level of CO2 distension and flow rate in directed tissue drying. In FIG. 23 insufflation tubing 410 is attached directly to auxiliary valve 34 and valve handle 35 is in the full open position. Auxiliary valve 334 is also in the full open position by means of valve handle 335 indicating a maximum insufflator flow rate into open valve 34 for directed tissue drying as CO2 gas will continue to escape from open valve 334 preventing the insufflator from achieving the pressure set point, which allows uninterrupted gas flow to continue. FIG. 24 illustrates a fully open valve 34 and a closed valve 334 indicating distension is achieved rapidly with no gas outflow allowed through closed auxiliary valve 334. The level of inflow and outflow, achieving insufflator pressure set point, level of distension and flow rate for directed tissue drying are controlled by positioning of valve handles 35 and 335 in the fully open, fully closed and partially open position on auxiliary valves 34 and 334.

In yet another embodiment for the control of CO2 distension and directed tissue drying, the configuration of valve 34 fully open and valve 334 fully closed as illustrated in FIG. 24 can also be used for directed tissue drying and level of distension by using trumpet valve 14 on handpiece 10 to suction excess CO2 gas out of the joint allowing a continuing inflow rate through open valve 34 and a continuing level of preferred distension, physician controlled by depressing trumpet suction valve 14.

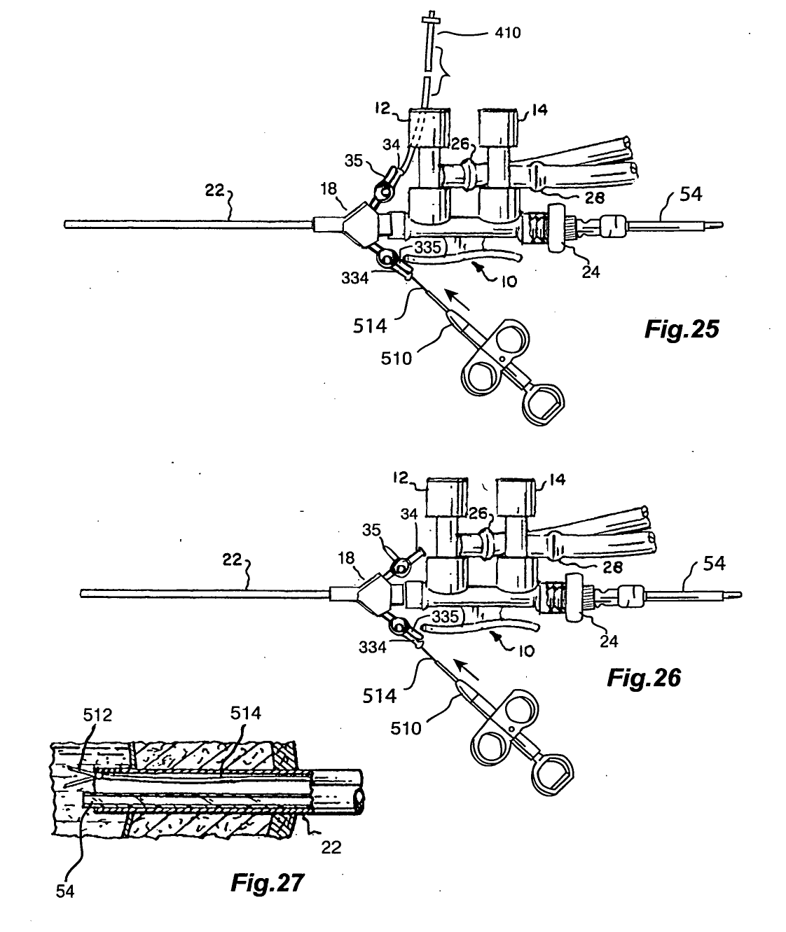

Use of the system atraumatic grasping device 510 for placement of a biocompatible tissue scaffold is illustrated in FIG. 25. This embodiment illustrates distension inflow from insufflator 400, with insufflation tubing 410 attached directly to auxiliary valve 34 and valve handle 35 is in the full open position. Auxiliary valve 334 is also in the full open position by means of valve handle 335 to accept the system 1 mm atraumatic grasper.

Insertion and use of flexible grasper 510 through open auxiliary valve 334 blocks the escape of gas through valve 334 and therefore valve 334 is essentially closed. Maintenance and control of distension in this configuration would allow insufflator 400 to reach the pressure set point to maintain the preferred level of distension as long as grasper 510 remained in valve 334.

Trumpet valve 14 on handpiece 10 can also be depressed to suction off excess gas.

FIG. 26 also illustrates use of atraumatic grasper 510 inserted in open valve 334, however valve 34 in this embodiment is closed. With both auxiliary valves not allowing gas flow, distension is controlled entirely by trumpet valves 12 and 14 on handpiece 10 as described previously in FIG. 22.

Insufflator 400 is connected to insufflation tubing 410 which is connected to the severed end of inflow/irrigation tubing 58 by means of a standard tubing connector. Trumpet valves 12 and 14 on handpiece 10 are operated in an identical manner to that when performing the lavage segment of the procedure. Shaft 514 of grasping device 510 is inserted into open auxiliary valve 334 on cannula 22. FIG. 27 illustrates a close up view of atraumatic grasper 510's shaft 514 inside cannula 22 with arthroscope 54. Grasper jaws 512 of grasper 510 are shown extended beyond the tip of cannula 22 and inside the joint space. Activation of the ring handle design opens and closes jaws 512 allowing placement of a biocompatible tissue scaffold to a targeted tissue site within a joint.

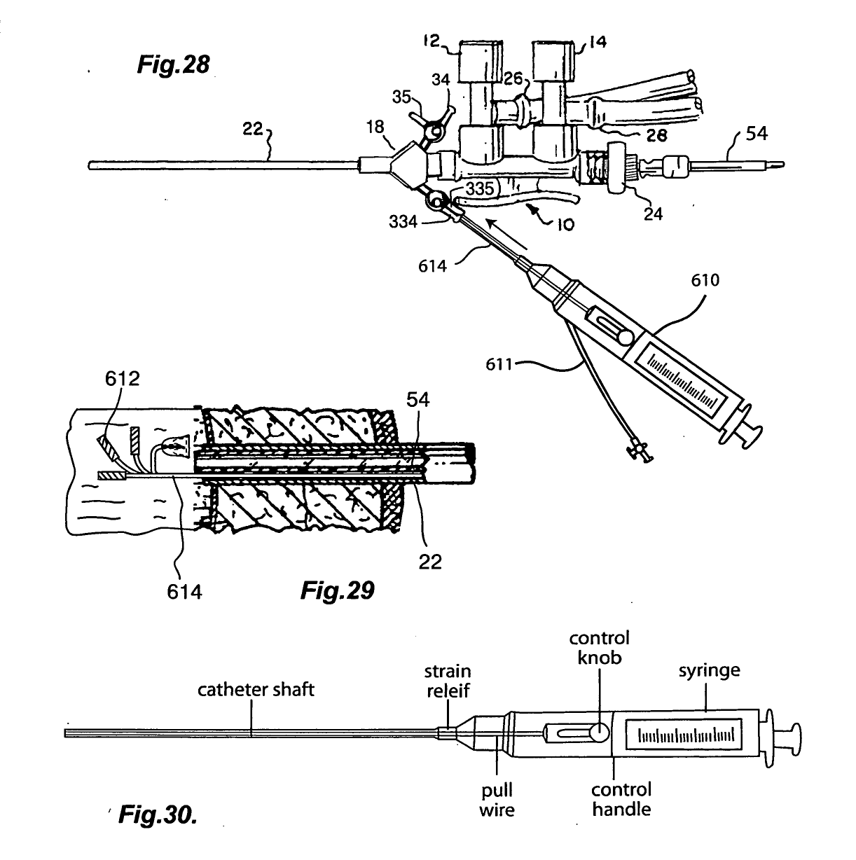

FIG. 28 and FIG. 29 illustrate system use of steerable catheter 610 for the placement of autologous regenerated cells to a dried tissue site or to a previously affixed biocompatible tissue scaffold. Catheter shaft 614 of steerable catheter device 610 is inserted into open auxiliary valve 334 on cannula 22 and advanced to the target site within the joint. FIG. 29 depicts cannula 22 as in the interior joint space and steerable catheter tip 612 is illustrated in various stages of deployment dependent upon tissue structure, location and size of area being treated, angle of approach to target site, and whether balloon isolation of said cell treatment area during cell infusion and placement would be advantages.

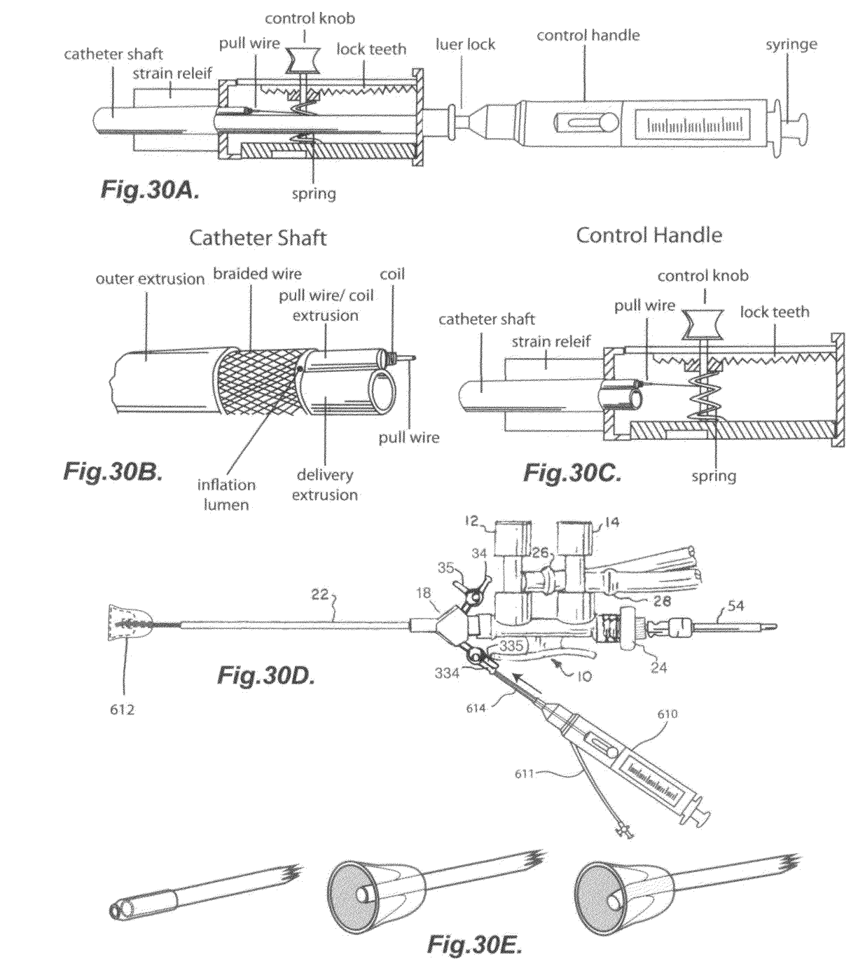

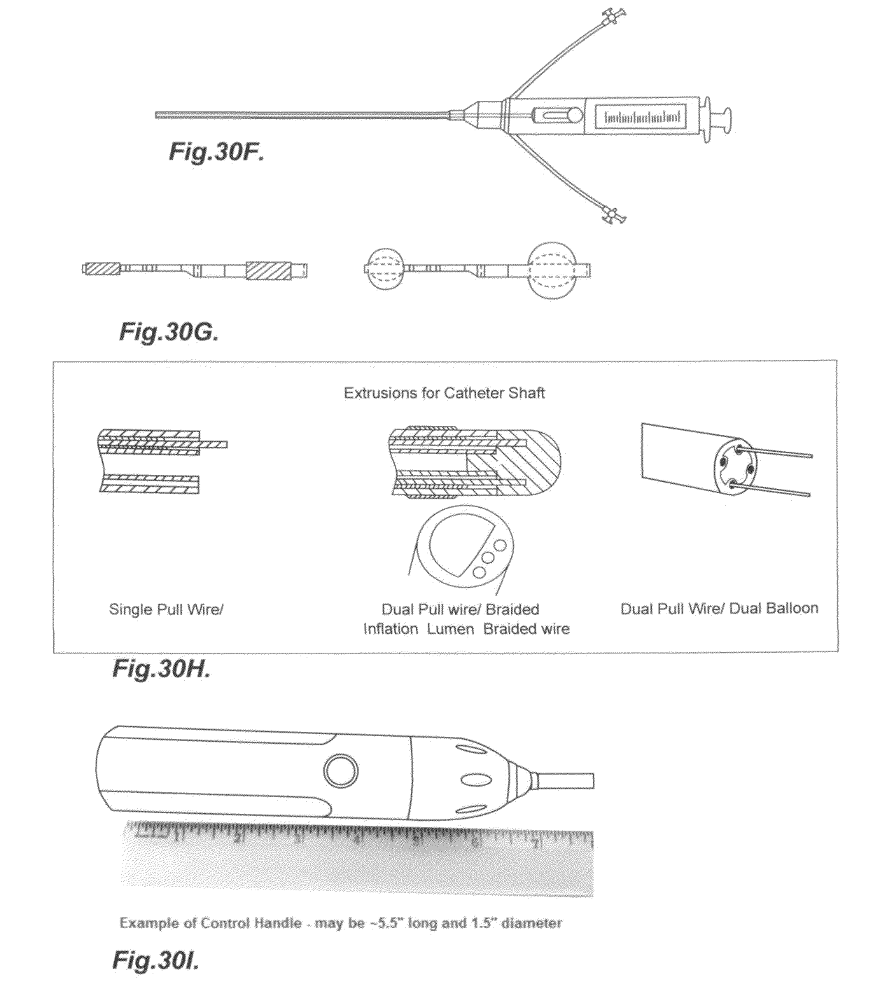

FIG. 30, 30A-30I illustrate different embodiments of cell delivery catheter 610 being no balloon, single and dual balloon configurations along with design, construction and materials generally found in steerable catheters.

A unique advantage of this system and its concept is the optical coupler, focusing mechanism, CCD camera, camera head and light source are all contained in one unit located off the sterile field. Additionally, the need for a separate light cable is also eliminated. The only optical/visualization component requiring sterilization is the arthroscope (54, 126 in FIG. 21) which contains integral illumination fibers. Other components of the arthroscopic lavage, tissue drying, tissue scaffold and regenerated cell delivery system used within the sterile field are disposable handpiece 10 and integrated irrigation and suction tubing 58 and 66, dual valve cannula 22 and loose body/biopsy cannula 100 along with sharp trocar 36, blunt obturator 48, rear adaptor 24, exchange rod 102, dilator shaft 202, biopsy instrument 110, atraumatic grasper 510 and cell delivery catheter 610.

To perform the arthroscopic lavage procedure segment, dual valve cannula 22 is placed in interior joint space 52 as illustrated in FIGS. 1 through 7. Irrigation and suctioning hoses 58 and 66 are permanently connected to irrigation and suction channels 26 and 28 on handpiece 10 and fiberoptic arthroscope 54 inserted through cannula 22. Alternately irrigation and suction is takes place until a clear picture is obtained on monitor 124.

Examination and diagnosis is performed while continuing to flush as needed by manipulating irrigation and suction trumpet valves 12 and 14 to maintain a clear operative field, with suction trumpet valve 14 operated to flush out loose bodies and debris as well as irritants typically found within a diseased joint space 52.

Generally up to 3 liters of sterile saline are used to perform the lavage and clean interior joint space 52. Removal of larger diameter loose bodies and use of larger diameter single-port instrumentation too large for cannula 22 use requires exchanging dual valve cannula 22 for loose body/biopsy cannula 100 as illustrated in FIGS. 13 through 20. With arthroscope 54 in place through loose body/biopsy cannula 100, a flexible instrument or device approximately 2 mm in diameter may be passed through valve 132 to perform biopsies with forceps jaws 112. Cutting, ablation and tissue contouring applications will also be accomplished through valve 132.

The embodiments of FIG. 19A through 19C permit irrigation, suction and use of biopsy instrument 110 under direct visualization while adding an additional auxiliary valve 232 in FIG. 19A, and valve 34′ in FIG. 19B and FIG. 19C. Also, FIG. 19C illustrates an embodiment transposing rear adaptor 24′ and auxiliary valve 34′ enabling flexible working end arthroscope 54′ to be used with a rigid or semi-rigid instrument 110′ inserted through valve 34′ at the rear of the device and in line with the central lumen, allowing greater versatility in instrument type and improved performance.

FIG. 19A is similar to FIG. 19C in the ability to use a flexible working end arthroscope through auxiliary valve 232 on the front of the device and a rigid or semi-rigid instrument inserted through at the rear of the device and in line with central lumen in handpiece 10 and cannula 100.

Use of system components on cart 120 or coming off the sterile field and connected to components on cart 120 for visualization require arthroscope 54/126 be attached to camera, camera head, optical coupler and light source housed in camera control module 122, which is in communication with digital still image recorder 123 and image footswitch control 125, which is in communication with monitor 124 for viewing the real time internal image seen by arthroscope 54. Components on cart 120, coming off the sterile field and connected to components on cart 120 used for fluid management of the lavage are inflow/irrigation tubing 58 (FIG. 21) spiked into hanging sterile saline bags, pressurized in irrigation pumps 140, pressure controlled by knobs 146 and gauges 148, powered by switches 142, allowing hose 138 from air compressor 130 to pressurize pumps 140.

Vacuum suction is supplied to handpiece 10 through suction tube 66, connected to suction canisters 136 which are in communication with suction console 132 by means of suction hose 134.

Following the lavage segment and sequencing into CO2 distension and directed tissue drying involves turning off compressor 130, switches 142 on irrigation pumps 140, and closing clamp 144 on irrigation tube 58.

FIG. 22 illustrates insufflator 400 on cart 120 attached to insufflation tubing 410, which is attached directly to the cut proximal end of inflow/irrigation tubing 58. The distal end of tubing 58 is still connected to handpiece 10. After clearing the distal end of tubing 58 of irrigation fluid a standard stepped tubing adaptor (not shown) is used to couple insufflation tubing 410 to tubing 58. No changes to suction tubing 66 connections are required. In this preferred embodiment inflow of CO2 gas, distension and directed tissue drying are physician controlled by trumpet valves 12 and 14 on handpiece 10 in an identical manner to their use during lavage. Depressing trumpet valve 12 now controls the inflow of CO2 gas, and depressing trumpet valve 14 controls the outflow of CO2 gas.

Following CO2 gas conversion and directed tissue drying, cells can be placed directly on a dried site or the site can be further prepared by the placement of a biocompatible tissue scaffold. In the preferred embodiment distension is controlled by valves 12 and 14 on handpiece 10, and as illustrated in FIG. 26 auxiliary valve 34 is closed and grasper 510 is inserted in open auxiliary valve 334 and advanced through cannula 22 into the interior joint space. Tissue scaffolds will be affixed directly to a dried site, or adhered with fibrin glue.

Placing autologous regenerated cells directly to a dried tissue site or on a tissue scaffold uses system steerable catheter 610. Catheter shaft 614 is inserted into open auxiliary valve 334 and advanced through cannula 22 into the interior joint space, and advanced and positioned at the target site.

Catheter tip 612 deflection is controlled by standard torque controls in the catheter handle and the use of 1 or 2 balloons, depending on anatomy, on catheter tip 612 allows precise cell delivery to a site isolated and protected by the inflated balloon or balloons.

Another embodiment using catheter based technology, not illustrated, will be for placement of tissue scaffolds. A flexible catheter of a similar dimension to catheter shaft 614 can be inserted and advanced through open auxiliary valve 334 and cannula 22 and into the interior joint space. This catheter device will deploy a rolled up tissue scaffold on a dried tissue site when the catheter control handle is activated. The catheter tip will also be steerable for precise scaffold placement. Scaffolds can also be affixed to a balloon deployed from within the distal lumen and be securely attached to the target site with a gentle but firm and uniform pressure.

The different segments of the arthroscopic lavage, tissue drying, tissue scaffold and regenerated cell placement procedure is performed using only a single, small entry port which minimizes trauma to the patient. The small single puncture only requires local anesthesia and is closed with steri-strips and covered with a simple bandage. Stitches are rarely required.

Thus there has been disclosed a unique and novel arthroscopic lavage, tissue drying, tissue scaffold and regenerated cell placement delivery system that uses only a single point of entry for performing diagnostic and therapeutic procedures.

The system includes a handpiece having valves for simultaneously manipulating a dual valve or loose body/biopsy cannula while performing irrigation and suctioning to wash out and remove any debris, loose bodies, as well as irritants contained within the joint. The system also includes a larger diameter loose body/biopsy cannula that can be easily exchanged by use of an exchange rod and dilator shaft. The loose body/biopsy cannula also allows for both irrigation and suction as well to remove larger loose bodies, biopsy to be performed with a biopsy instrument inserted through an auxiliary ball valve, and permits the use of larger diameter instruments and devices when necessary. In addition, both dual valve and loose body/biopsy cannula have the ability to separately infuse medication and remove sterile synovial fluid through the additional stopcock valve located on couplings 18 and 118. In addition, the conversion from saline fluid inflow to CO2 gas inflow to maintain distension and perform directed tissue site drying is easily and quickly accomplished by simply shutting down the irrigation circuit and connecting insufflation tubing 410 to inflow/irrigation tubing 58 with a standard stepped tubing connector. With insufflator 400 turned on, the depressing of trumpet valve 12 on handpiece 10 now controls the inflow of CO2 gas, and depressing trumpet valve 14 controls CO2 outflow.

Using system specific instrumentation and steerable catheters allows for the accurate and precise placement of biocompatible tissue scaffolds, and the accurate and precise placement of regenerated cells. The incorporation of balloon technology in system catheters allows for isolating tissue target sites during cell placement, and the precise placement and attachment of tissue scaffolds, aided by the gentle pressure of the balloon over the entire surface area of the target tissue site and the scaffold being affixed.

This invention is not to be limited by the embodiment shown in the drawings and described in the description which is given by way of example and not of limitation, but only in accordance with the scope of the appended claims.

What is claimed is:

1. A multi-function arthroscopic lavage, tissue drying, biocompatible tissue scaffold and regenerated cell placement delivery system for diagnostic examination and therapeutic treatment of joint diseases permitting entry, visualization, irrigation, suction, tissue drying, tissue scaffold and regenerated autologous cell placement through a single, small portal comprising;
a handpiece having a central passageway;
irrigation and suction channels connected to said handpiece;
irrigation and suction valves in said handpiece for intermittent control of irrigation fluid and suctioning; said irrigation and suction valves also used for intermittent control of inflow and suctioning of CO2 or other ‘dry’ gas;
a cannula;
a coupling for attaching said cannula to said handpiece;
entry instruments for inserting said cannula attached to said disposable handpiece into said joint;
an arthroscope for insertion through said handpiece and said cannula after removal of said entry instruments for visualization of the interior joint space, examination, and diagnosis;
said handpiece constructed for manipulating said cannula in a joint space while alternately irrigating and suctioning by operation of said irrigation and suction valves;
a means of conversion from a ‘wet’ sterile saline fluid cleaning and distension media to a ‘dry’ CO2 or other ‘dry’ gas distension media;
a means of directed tissue drying of specific tissue, structures and areas within a joint;
a means to accurately place a biocompatible tissue scaffold to a dried tissue site;
a means to accurately place regenerated autologous cells to a specific dried tissue site, structure and area within a joint;
a means to accurately place regenerated autologous cells to a previously affixed tissue scaffold;
whereby said joint may be efficiently and conveniently examined, diagnosed, thoroughly cleaned, dried, and treated by the placement and adherence of regenerated autologous cells to a specific dried tissue site, structure and/or area within a joint through a small single entry port;
whereby said joint may be efficiently and conveniently examined, diagnosed, thoroughly cleaned, dried, and treated by the placement and adherence of a biocompatible tissue scaffold to a specific dried tissue site, structure and/or area within a joint, followed by the precise placement and adherence of regenerated autologous cells to said scaffold through a small single entry port.

2. The system according to claim 1 in which said handpiece is a disposable handpiece

3. The system according to claim 1 in which said irrigation and suction valves are trumpet valves in communication with a central passageway through said disposable handpiece;
an embodiment (not shown) in which said irrigation and suction valves are spring loaded and in communication with a central passageway through said disposable handpiece, and said valves are both accessed by a single slide control in communication with both the irrigation and suction valves on said handpiece

4. The system according to claim 1 including 2 auxiliary valves in said coupling providing access to said space through said cannula.

5. The system according to claim 1 in which said arthroscope, with integrated illumination fibers is directly connected to an optical coupler, camera head, CCD or CMOS camera, and light source; said arthroscope having an outside working diameter that is equal to or less than approximately one-half the inside diameter of said cannula.

6. The system according to claim 5 in which said coupling for attaching said cannula to said handpiece is a coupling with said cannula attached; said handpiece having a socket for receiving said coupling whereby said cannula may be quickly attached or removed from said disposable handpiece

7. The system according to claim 6 in which said cannula is a dual valve swivel cannula which allows multiple locking positions of the cannula in relation to the handpiece for the insertion and advancement of small diameter instruments, devices and catheters into and through the auxiliary valves and into the central lumen of the cannula for superior positioning and maneuvering within and around the joint space.

8. The system according to claim 6 in which said cannula is a larger diameter ‘loose body’ removal and ‘biopsy’ cannula.

9. The system according to claim 8 in which said instruments comprise a sharp trocar and blunt obturator for inserting said cannula into said joint space.

10. The system according to claim 9 in which said cannula contains 2 auxiliary valves having angled channels for introducing medication, biocompatible tissue scaffolds, autologous regenerated cells, surgical instruments, devices and catheters.

11. The system according to claim 8 in which said cannula is a larger diameter ‘loose body’ removal and ‘biopsy’ cannula that is exchanged by use of an exchange rod.

12. The system according to claim 11 including a tapered dilator shaft for use with said exchange rod for gentle expansion of said small portal to facilitate insertion of said larger diameter ‘loose body’ removal and ‘biopsy’ cannula.

13. The system according to claim 12 in which said ‘large diameter’ loose body removal and ‘biopsy’ cannula comprises a larger central channel, and a larger auxiliary valve having an angled channel for introducing larger diameter surgical instruments and devices.

14. The system according to claim 1 in which said arthroscope comprises; a 30,000 pixel fiberoptic image bundle of approximately 1.2 mm diameter, illumination fibers, dual element distal lens, stainless steel sheath at a working end, a scope lock for securing said arthroscope to said handpiece and separate image and illumination plug-ins at a proximal end for connection to an optical coupler, an arthroscopic camera and a light source.
an embodiment in which said arthroscope comprises; a CCD, CMOS, or other type of miniature image sensor mounted at the distal end of the arthroscope.
an embodiment in which said arthroscope has a flexible working end for traversing a cannula with an angled bend at the tip
an embodiment in which said arthroscope utilizes LED illumination

15. The system according to claim 1 including irrigation channels in said handpiece; an irrigation tube connected to irrigation, pressurized and controlled solution storage containers; and a compressor for providing said irrigation fluid pressure through said irrigation tube to said irrigation channel for control by said irrigation valve.

16. The system according to claim 15 including a vacuum suction device connected to said disposable handpiece; said suction device comprising a suction hose connected to said disposable handpiece; collection canisters connected to a suction console; said suction console containing a vacuum pump connected to said collecting canisters for drawing fluid and loose material from within the joint through said disposable handpiece; said suction being controlled by said suction valve in said disposable handpiece.

17. The system according to claim 14 including a high resolution monitor connected to said arthroscopic camera; and a recording device for documenting the arthroscopic procedure.

18. The system according to claim 17 in which said recording device is a video recording device.

19. The system according to claim 17 in which said recording device is capable of producing video prints.

20. The system according to claim 17 including a self-contained mobile cart for storing and transporting said video camera and light source, said high resolution monitor, said recording device, said compressor, said vacuum pump, said collecting canisters, and said irrigation solution pumps, said insufflator, whereby said system may be easily transported to an operation site and positioned outside of the sterile field for convenient viewing.

21. The system according to claim 20 in which an optical coupler, focusing mechanism, video camera, camera head and light source are off the sterile field for ease of use and minimize the components in the sterile field; whereby an interior space of said joint may be visualized, examined, diagnosed, lavaged by alternate irrigation and suction in preparation for further treatment.

22. The system according to claim 1 including a coupling on an end of said disposable handpiece opposite said cannula, said coupling having a central main channel and an auxiliary angled channel, whereby an arthroscope and an instrument or other device may be inserted through said cannula for simultaneous observation and treatment of said joint space.

23. The system according to claim 1 including a cannula attaching to the front of said disposable handpiece having a central main channel and 2 auxiliary angled channels, whereby an arthroscope and an instrument or other device may be inserted through said cannula for simultaneous observation and treatment including biocompatible tissue scaffold and regenerated autologous cell placement within said joint.

24. The system according to claim 1 including a cannula attaching to the front of said disposable handpiece having a central main channel and an auxiliary angled channel, whereby an arthroscope and a larger diameter biopsy instrument or other device may be inserted through said cannula for simultaneous observation and treatment including tissue specimen biopsy, biocompatible tissue scaffold and regenerated autologous cell placement within said joint.

25. The system according to claim 23 including 2 auxiliary valves on said cannula; said angled channels passing through said cannula for the separate insertion of medication, an instrument, device or catheter, biocompatible tissue scaffold and regenerated autologous cell placement within said joint.

26. The system according to claim 25 including auxiliary valves on said coupling; said angled channels passing through said auxiliary valves.

27. The system according to claim 26 in which said arthroscope is inserted through said central main channel and said medication, tissue scaffold, autologous cell placement, instruments, devices and catheters are inserted through said auxiliary channels.

28. The system according to claim 27 in which said medication, tissue scaffold, autologous cell placement, instruments, devices and catheters are inserted through said central main channel and said arthroscope is inserted through said auxiliary angled channel.

29. The system according to claim 1 in which said auxiliary valve on cannula attaching to the front of said disposable handpiece is open and attached to a CO2 gas insufflator by means of tubing, and the other auxiliary valve remains fully or partially open to maintain distension and facilitate directed tissue drying.

30. The system according to claim 1 in which a CO2 gas insufflator is attached directly to the inflow/irrigation tubing attached to said handpiece. Depressing the irrigation trumpet valve on said handpiece allows the flow of regulated CO2 gas through said irrigation valve directly in to central lumen of said handpiece and into and through the central lumen of said cannula attached to said handpiece and directly into the interior joint space.

31. The system according to claim 30 in which the distension and directed tissue site drying of said joint is controlled and regulated by said irrigation trumpet valve for CO2 gas inflow, and by said suction trumpet valve for CO2 gas outflow.

32. The system according to claim 4 in which autologous regenerated cells are placed into said joint in a global fashion by means of direct injection using a syringe attached to said auxiliary valve on said cannula.

33. The system according to claim 1 in which a small 1 mm grasping instrument or device is inserted into an open auxiliary valve on said cannula and advanced into and through said central lumen of said cannula and into the interior joint space to accurately affix a biocompatible tissue scaffold to a dried tissue under direct visualization.

34. The system according to claim 1 in which a small 1 mm catheter is inserted and advanced into and through an open auxiliary valve on said cannula, into the interior joint space and guided to a dried tissue site, or previously affixed biocompatible tissue scaffold, for the accurate placement of autologous regenerated cells under direct visualization. Said system catheter incorporates a torque handle for manipulation and control of the catheter tip for precise placement and layering of cells on said dried tissue site or tissue scaffold.
